(19) United States
(12) Patent Application Publication
(10) Pub. No.: US 2025/0082808 A1
(43) Pub. Date: Mar. 13, 2025

(54) Digital Autoreader using Electronic Based Sensors for Monitoring of Sterilization Processes

(71) Applicant: O&M Halyard, Inc., Mechanicsville, VA (US)

(72) Inventors: Anthony S. Spencer, Woodstock, GA (US); Marshall R. Dean, Cumming, GA (US); Peymaun Ghafouri-Kia, Alpharetta, GA (US); Alexander Fromholtz, Verona, NJ (US); Alexander Gorshkov, Fort Wayne, IN (US); Brian E. Lin, Cumming, GA (US); Samuel G. Israel, Alpharetta, GA (US); Sylvie Dufresne, Quebec (CA)

(21) Appl. No.: 18/825,130

(22) Filed: Sep. 5, 2024

Related U.S. Application Data
(60) Provisional application No. 63/581,287, filed on Sep. 8, 2023.

(51) Int. Cl. A61L 2/28

(52) U.S. Cl. CPC A61L 2/28

(57) ABSTRACT

A digital autoreader is provided. The digital autoreader includes a housing including an interior and an exterior; a well for receiving a process challenge device, the process challenge device including at least one indicator, the well including an opening and a block for retaining the process challenge device, wherein the block is located within the interior of the housing; a first sensor located within the interior of the housing and positioned adjacent to the block of the well, the first sensor configured to read a variable of the at least one indicator of the at least one indicator of the process challenge device and determine whether the process challenge device has been adequately sterilized; and an interface to alert a user if the process challenge device has been adequately sterilized.

### FIELD OF THE INVENTION

The present invention relates generally to a system used to verify the effectiveness of a sterilization process.

### BACKGROUND

Various systems for determining the efficacy of sterilization processes are known in the art. There are several types of indicators capable of determining the efficacy of sterilization processes used in the field, each providing differing means to provide the required assurance to the user that the appropriate processing requirements were met which would result in proper sterilization. The sterilization process's efficacy must be routinely monitored or otherwise validated to assure the safety of any product to be sterilized (e.g., medical devices, instruments, etc.) post-sterilization. To this end, a process challenge device (PCD) is typically provided to monitor the sterilization and assess the overall effectiveness of the sterilization by providing a challenge to the sterilization process that is equal to or greater than the challenge posed by the most difficult-to-sterilize item used in the particular field or setting. To this end, PCDs can include specific types of sterilization monitoring means such as chemical and biological indicators.

A chemical indicator (CI) is a non-biological indicator test system that reveals a change in one or more pre-defined process variables based on a chemical or physical change resulting from exposure to a specific sterilization process. The chemical or physical change often results in the CI visually changing such that a user may determine whether the change has occurred or the quality or degree of the change that has occurred as a result of the exposure to the process. For example, the CI may show a color change or a change in the shape of a marking on the chemical indicator. However, to observe this visual change within the CI, the CI must be removed from the PCD to be read by a user. Further, the visual change in the CI can often be misread by the user, which may result in improper verification of the status of the sterilization processing.

A biological indicator (BI) provides sterility assurance by using biological systems that indicate that the efficacy of a sterilization process has been successfully achieved. A BI contains a known population of highly resistant microorganisms, usually bacterial spores, that are specific to the sterilization process being monitored and meet pre-determined resistance characteristics for the specific sterilization process. The microorganism is typically inoculated on a carrier, and the inoculated carrier can be placed in packaging permeable to the sterilization process. The inoculated carrier can also be placed within a packaging that also contains the growth medium in a separate compartment or a sealed glass ampoule as well as a sterile barrier to prevent adventitious contamination. This type of BI is called a self-contained biological indicator (SCBI). In some SCBIs, the microorganism is not placed on a carrier but inoculated directly on an interior portion of the container.

However, to determine if the microorganisms have been sterilized after exposure to a sterilization cycle, the SCBI needs to be removed from the PCD, and manually activated to ensure that the microorganisms contained in the BI come into contact with the growth medium. This action must take place without permitting external contamination of microorganisms and growth medium thereby maintaining a sterile barrier. Once activated, a biological indicator needs to be placed in an incubator that has the appropriate incubating conditions, such as temperature and time, for any microorganisms present to grow. Each step may be prone to error and result in contamination of the BI which may result in improper verification of the status of the sterilization processing.

While each of these types of indicators provides its own unique advantages, both types of indicators need to be manipulated and/or read by end users in the healthcare facility or dental sterilization processing area. This requirement may lead to a misreading of the indicator(s) or error(s) in recording the indicator results. A misreading may also lead to false interpretation of the indicator results, and improper verification of the status of the sterilization processes thereby endangering patients. Thus, there is a need for a system that can utilize the unique advantages of each of the types of indicators while avoiding the potential for inaccuracies or misreading of the different indicators. In addition, there is a need for separate documentation of each indicator component of the PCD after processing and evaluation. Specifically, an integrated process challenge device and a digital autoreader that addresses these issues is desirable. For example, it is desirable to have automatic documentation of the results for each type of indicator.

### SUMMARY OF THE INVENTION

Objects and advantages of the invention will be set forth in part in the following description, or may be obvious from the description, or may be learned through practice of the invention.

In accordance with one embodiment of the present invention, a digital autoreader for determining efficacy of a sterilization process is provided. The digital autoreader includes a housing including an interior and an exterior; a well for receiving a process challenge device, the process challenge device including at least one indicator, the well including an opening and a block for retaining the process challenge device, wherein the block is located within the interior of the housing; a first sensor located within the interior of the housing and positioned adjacent to the block of the well, the first sensor configured to read a variable of the at least one indicator of the at least one indicator of the process challenge device and determine whether the process challenge device has been adequately sterilized; and an interface to alert a user if the process challenge device has been adequately sterilized.

In one aspect, the at least one indicator includes a chemical indicator, wherein the first sensor includes a chemical indicator reader.

In another aspect, the chemical indicator reader includes a camera.

In still another aspect, the digital autoreader further includes a processor including a machine learning algorithm programmed therein, the machine learning algorithm configured to read an output of the at least one indicator and determine if a change in an appearance of the at least one indicator indicates that the process challenge device has been adequately sterilized.

In a further aspect, the at least one indicator includes a biological indicator, wherein the first sensor includes a biological indicator reader, wherein the biological indicator reader includes a photoemitter and a photodetector.

In yet another aspect, the digital autoreader further includes a second sensor located within the interior of the housing and positioned adjacent to the block of the well, the second sensor configured to read a variable of a second indicator and determine whether the process challenge device has been adequately sterilized, wherein the second sensor is located at a location different from where the first sensor is located.

In another further aspect, the interior of the housing includes a crushing mechanism configured to crush a container within the process challenge device, the container having a growth medium housed therein.

In yet another further aspect, the interior of the housing includes a mixing mechanism.

In still yet another further aspect, the mixing mechanism is arranged adjacent to an end of the container such that the mixing mechanism can saturate a biological culture sample of the process challenge device with the growth medium when the container is crushed.

In a still further aspect, the interior of the housing includes a heating element for incubating the biological culture sample and the growth medium at a desired temperature.

In yet a further aspect, the first sensor is configured to detect at least one of a volume of the growth medium contained within the container or a saturation level of a biological culture sample and the growth medium.

In another aspect, the digital autoreader further includes a scanner mounted on the exterior of the housing, wherein the scanner includes at least one of a barcode scanner or a QR code scanner.

In yet another aspect, the first sensor includes at least one of a camera, barcode scanner, a QR code scanner, an RFID reader, or an NFC reader.

In still another aspect, the block of the well includes a locking mechanism, the locking mechanism configured to selectively lock and release the process challenge device within the well.

In a further aspect, the digital autoreader includes a plurality of wells for receiving a plurality of process challenge devices.

In yet another aspect, the interface displays information corresponding to the process challenge device inserted within the well.

In another further aspect, the interface includes a tab corresponding to the process challenge device inserted within the well, the tab including at least one of a result icon communicating whether the process challenge device has been adequately sterilized or a timer corresponding to the time required to determine whether the process challenge device has been adequately sterilized.

In yet another further aspect, the well includes a locating feature for correctly orienting the process challenge device within the well.

In a still further aspect, the at least one indicator includes a biological indicator, biological indicator includes at least one of a gas sensor or a conductivity sensor.

In another embodiment, the present disclosure is directed to a digital autoreader for determining efficacy of a sterilization process. The digital autoreader includes a housing including an interior and an exterior; a well for receiving a process challenge device including a first indicator and a second indicator, the well including an opening and a block for retaining the process challenge device, wherein the block is located within the interior of the housing; a first sensor located within the interior of the housing and positioned adjacent to the block of the well, the first sensor configured to read a variable of the first indicator and determine whether the process challenge device has been adequately sterilized; a second sensor located within the interior of the housing and positioned adjacent to the block of the well, the second sensor configured to read a variable of the second indicator and determine whether the process challenge device has been adequately sterilized, wherein the second sensor is located at a location different from where the first sensor is located; and an interface to alert a user if the process challenge device has been adequately sterilized.

In still another embodiment, the present disclosure is directed to a method for determining efficacy of a sterilization process. The method includes providing a digital autoreader including: a housing including an interior and an exterior, a well for receiving a process challenge device, the process challenge device including at least one indicator, the well including an opening and a block for retaining the process challenge device, wherein the block is located within the interior of the housing, a first sensor located within the interior of the housing and positioned adjacent to the block of the well, the first sensor configured to read a variable of the at least one indicator of the at least one indicator of the process challenge device and determine whether the process challenge device has been adequately sterilized, and an interface to alert a user if the process challenge device has been adequately sterilized. The method further includes the steps of inserting the process challenge device within the well; and operating the digital autoreader to determine if the process challenge device has been adequately sterilized.

In yet another embodiment, the present disclosure is directed to a system for determining efficacy of a sterilization process. The system includes an integrated process challenge device including: a shell including an interior and an exterior, a compartment defined by the interior and exterior of the shell, the compartment shaped to retain a biological indicator, and an opening located on the exterior of the shell adjacent to the compartment, wherein the opening allows a sterilant to enter and exit the compartment. The system further includes a digital autoreader including: a housing including an interior and an exterior, a well for receiving the integrated process challenge device, the well including an opening and a block for retaining the integrated process challenge device, wherein the block is located within the interior of the housing, a biological indicator reader, the biological indicator reader configured to read a variable on the biological indicator and determine whether the integrated process challenge device has been adequately sterilized, a crushing mechanism configured to crush a container within the biological indicator, the container having a growth medium housed therein, and an interface to alert a user if the integrated process challenge device has been adequately sterilized.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a perspective view of an embodiment of an integrated process challenge device according to the present disclosure;

FIG. 2 is an exploded view of an embodiment of the integrated process challenge device of FIG. 1 according to the present disclosure;

FIG. 3a is a rear view of the integrated process challenge device of FIGS. 1-2 according to the present disclosure;

FIG. 3b is a front view of the integrated process challenge device of FIGS. 1-2 according to the present disclosure;

FIG. 4 is a front view of an example embodiment of a chemical indicator according to the present disclosure;

FIG. 5a is an exploded perspective view of a biological indicator according to the present disclosure;

FIG. 5b is a front perspective view of a biological indicator according to the present disclosure;

FIG. 6 is a side cutaway view of an embodiment of an integrated process challenge device according to the present disclosure, particularly illustrating a biological indicator placed within the integrated process challenge device;

FIG. 7 is a side cutaway view of another embodiment of a biological indicator placed within an integrated process challenge device according to the present disclosure;

FIG. 8a is a perspective view of an embodiment of a compressible region of an integrated process challenge device according to the present disclosure;

FIG. 8b is a side view of an embodiment of a compressible region of an integrated process challenge device according to the present disclosure;

FIG. 8c is a side view of an embodiment of a compressible region of an integrated process challenge device, particularly illustrating the compressible region being ruptured inwardly according to the present disclosure;

FIG. 9a is a top view of an embodiment of an integrated process challenge device according to the present disclosure, particularly illustrating locating features defined on the integrated process challenge device;

FIG. 9b is a bottom view of an embodiment of an integrated process challenge device according to the present disclosure, particularly illustrating locating features defined on the integrated process challenge device;

FIG. 9c is a side view of an integrated process challenge device according to the present disclosure, particularly illustrating locating features defined on the integrated process challenge device;

FIG. 10a is a partial perspective view of an embodiment of an integrated process challenge device according to the present disclosure, particularly illustrating a first flow path into the interior of the integrated process challenge device;

FIG. 10b is a partial perspective view of an embodiment of an integrated process challenge device according to the present disclosure, particularly illustrating a second flow path into the interior of the integrated process challenge device;

FIG. 10c is a partial perspective view of an embodiment of an integrated process challenge device according to the present disclosure, particularly illustrating a third flow path into the interior of the integrated process challenge device;

FIG. 11 is a partial back view of an embodiment of an integrated process challenge device;

FIG. 12 is a back view of an embodiment of an integrated process challenge device with a thermal indicating label according to the present disclosure;

FIG. 13a is a perspective view of an embodiment of a coding label that can be applied to an integrated process challenge device according to the present disclosure;

FIG. 13b is a perspective view of an embodiment of a coding label that can be applied to an integrated process challenge device according to the present disclosure;

FIG. 14 is a perspective view of an embodiment of an integrated process challenge device according to the present disclosure, particularly illustrating ports capable of facilitating flow of a sterilant into and from the interior of the integrated process challenge device;

FIG. 15 is a perspective view of an embodiment of an integrated process challenge device according to the present disclosure, particularly illustrating a support formed with the integrated process challenge device;

FIG. 16 is a perspective view of an embodiment of a digital autoreader according to the present disclosure;

FIG. 17 is a perspective view of the digital autoreader of FIG. 16 according to the present disclosure, particularly illustrating the interior of the digital autoreader;

FIG. 18 is a side view of an embodiment of a well of a digital autoreader according to the present disclosure;

FIG. 19 is a perspective view of an embodiment of a biological indicator reader according to the present disclosure;

FIG. 20a is a side view of an embodiment of a crushing mechanism according to the present disclosure;

FIG. 20b is an exploded perspective view of an embodiment of a crushing mechanism according to the present disclosure;

FIG. 20c is a bottom view of an embodiment of a crushing mechanism according to the present disclosure;

FIG. 21a is a side view of the crushing mechanism of FIGS. 20a-20c being used in conjunction with a process challenge device according to the present disclosure;

FIG. 21b is a bottom view of the crushing mechanism of FIGS. 20a-20c being used in conjunction with a process challenge device according to the present disclosure;

FIG. 22 is a side view of another embodiment of a crushing mechanism according to the present disclosure;

FIG. 23 is a side view of an embodiment of a mixing element according to the present disclosure;

FIG. 24a is a side view of an embodiment of a locking mechanism of a digital autoreader according to the present disclosure, particularly illustrating the locking mechanism locking a process challenge device within the digital autoreader;

FIG. 24b is a side view of an embodiment of a locking mechanism of a digital autoreader according to the present disclosure, particularly illustrating the locking mechanism being unlocked;

FIG. 25 is a top view of a well of an embodiment of a digital autoreader according to the present disclosure, particularly illustrating complementary locating features provided on the digital autoreader;

FIG. 26 is a perspective view of an embodiment of an expansion hub provided with a digital autoreader according to the present disclosure;

FIG. 27a is an illustration of an embodiment of an interface of a digital autoreader according to the present disclosure;

FIG. 27b is an illustration of an embodiment of an interface of a digital autoreader according to the present disclosure, particularly illustrating a detailed information tab;

FIG. 28 is a flow chart illustrating a method for determining efficacy of a sterilization process according to the present disclosure;

FIG. 29 is a flow chart illustrating a method for determining efficacy of a sterilization process according to the present disclosure; and

FIG. 30 is a flow chart illustrating a method for determining efficacy of a sterilization process according to the present disclosure; and

FIG. 31 is a flow chart illustrating a method for determining efficacy of a sterilization process according to the present disclosure.

### DETAILED DESCRIPTION OF THE INVENTION

Reference will now be made in detail to one or more embodiments of the invention, examples of which are illustrated in the drawings. Each example and embodiment is provided by way of explanation of the invention and is not meant as a limitation of the invention. For example, features illustrated or described as part of one embodiment may be used with another embodiment to yield still a further embodiment. It is intended that the invention include these and other modifications and variations as coming within the scope and spirit of the invention.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

Generally speaking, the present disclosure is directed to systems and methods for determining the efficacy of a sterilization process or verification of the sterilization process conditions. The system can include a process challenge device (PCD) having an integrated configuration that allows for one or more (or at least one) indicator to be provided therein. The system can also include a digital autoreader which includes more than one sensor that can read variables if more than one indicator is provided with the PCD. The system is capable of determining whether the indicator(s) contained within the integrated PCD have been adequately sterilized without requiring the integrated PCD to be opened directly or otherwise accessed after undergoing sterilizing conditions within a sterilization system. For example, the digital autoreader may be able to determine whether biological activity in a biological indicator has been inactivated, and/or if specific sterilization parameters have been met that would indicate that the integrated PCD and the medical instrument load within the sterilizer have been sterilized to a sufficient standard, typically the required Sterility Assurance Level (SAL) standard.

Further, the system is capable of performing multiple types of tests to determine the adequacy of the sterilization delivered by the sterilization system. For example, the integrated process challenge device can include multiple distinct compartments which help facilitate the necessary sterilizing conditions for distinct types of indicators such as chemical indicators (CIs) or biological indicators (BIs). Moreover, the digital autoreader can include multiple distinct sensors that are capable of reading the distinct types of indicators like the CIs and BIs. The disclosure may also include methods which take advantage of the unique features and benefits provided by using the integrated PCD and the digital autoreader. Thus, the present disclosure provides many unique advantages over the previous systems and methods known in the art. Each of these advantages and features will be discussed in greater detail hereinbelow with reference to the figures.

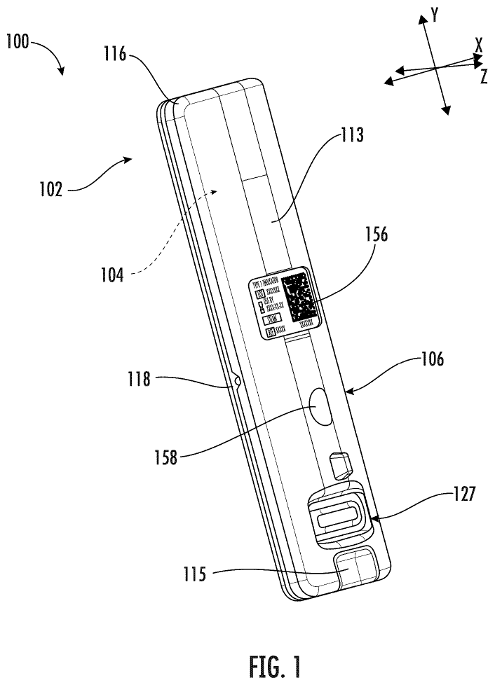

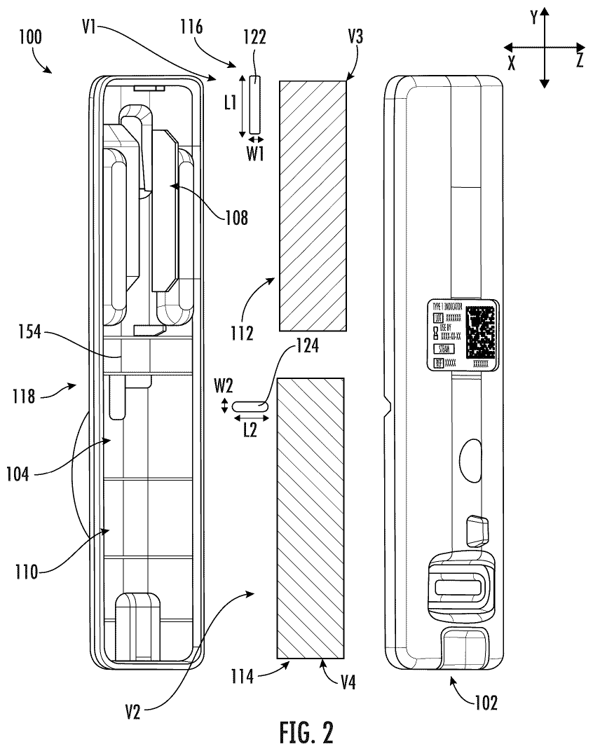

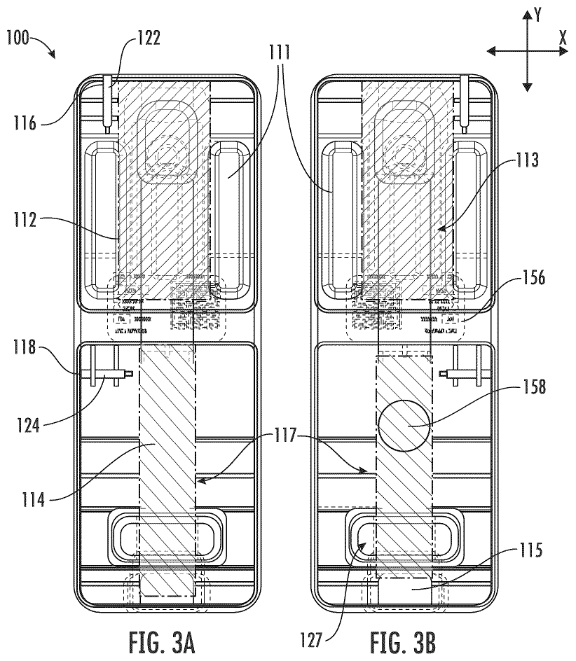

Referring now to FIG. 1, a perspective view of an embodiment of an integrated process challenge device (PCD) 100 is provided according to the present disclosure. FIG. 1 and other views and embodiments may be placed within a Cartesian coordinate system having an X-direction, a Y-direction, and a Z-direction. Now referring particularly to FIG. 1, the integrated PCD 100 includes a shell 102 having an interior 104 (see FIG. 2) and an exterior 106. The shell 102 may be formed out of a variety of materials such as a combination of materials with physical or chemical properties that allow for specific functions with the integrated PCD. In particular, a material with optical clarity or translucence may allow for visualization of a chemical indicator (CI) by an end-user through a first window region 113 or a second window region 115 via a digital autoreader. Further, the material may be selected to provide properties to provide for efficient heating of a biological indicator (BI) through the shell 102. Example materials may include polypropylene, polyethylene, polyester, such as Polyethylene Terephthalate (PET), polycarbonate (PC), Polystyrene, Polysulfone resin, Poly (tetrafluoroethylene). The aforementioned polymer materials may also include polymer modifiers or fillers such as, but not limited to alumina, silica, boron nitride, aluminum nitride, silicon carbide, graphite, diamond, graphene, carbon nanotubes, carbon fiber. Within the interior 104 of the shell 102, a first compartment 108 that may house a first indicator (such as a chemical indicator (CI) 112) and a second compartment 110 that may house a second indicator (such as a biological indicator (BI) 114) are provided. (See FIGS. 2-3). Along the exterior 106 of the shell 102, a first window region 113 and a second window region 115 are provided. Further, a first opening 116 and a second opening 118 are provided on the exterior 106 of the shell 102. Various labels may also be placed on the exterior 106 such as a coding label 156 and an indicator label 158. Each of these features will be discussed in greater detail with reference to the CI 112 and the BI 114. However, it is to be understood that the first indicator is not limited to the CI 112 and the second indicator is not limited to the BI 114. Moreover, while the shell 102 includes both the first compartment 108 and the second compartment 110, the shell 102 is not required to house multiple indicators therein.

Referring now to FIG. 2, an exploded view of an embodiment of the integrated process challenge device of FIG. 1 is provided according to the present disclosure. As shown, the first compartment 108 and the second compartment 110 are each defined by the interior 104 and the exterior 106 of the shell 102. Further, as shown, the first compartment 108 may be shaped or sized such that when the CI 112 is positioned alongside the first compartment 108 within the interior 104, the first compartment 108 is shaped to retain the CI 112. Like the first compartment 108, the second compartment 110 may be shaped or sized to retain the BI 114. For example, the first compartment 108 may have a total volume V1 that is larger than a volume V3 of the CI 112. Further, the second compartment 110 may have a total volume V2 that is larger than a volume V4 of the BI 114.

In addition, the first compartment 108 and the second compartment 110 may be distinctly separated by a barrier 154 formed from the shell 102. The barrier 154 may be formed between the first compartment 108 and the second compartment 110 such that the first compartment 108 and the second compartment 110 are isolated from each other. Specifically, the barrier 154 can prevent the flow of fluid, e.g., sterilant, between the first compartment 108 and the second compartment 110. By preventing the flow of sterilant between the first compartment 108 and the second compartment 110, the conditions within the first compartment 108 and the second compartment 110 may be individually designed such that specific sterilizing resistance conditions may be created unique to the first compartment 108 and the second compartment 110 when the PCD 100 is placed within a sterilization system. For example, the first opening 116 and the second opening 118 may each be sized such that a sterilant may enter and exit the first and second compartments 108, 110. Further, the first opening 116 may be sized such that only a specified amount of sterilant can enter the first compartment 108, and the second opening 118 may be sized such that only a specified amount of sterilant can enter the second compartment 110. By sizing the openings 116, 118 in such a manner, the sterilizing resistance conditions may be individualized for the first compartment 108 and the second compartment 110, respectively. For example, the sterilizing resistance conditions of the first compartment 108 may be made to be greater than the second compartment 110. This difference would be particularly advantageous if a CI 112 is placed within the first compartment 108 and a BI 114 is placed in the second compartment 110. This is because if the resistance to sterilizing conditions for a CI 112 is lower than the resistance to sterilizing conditions for a BI 114, the CI 112 may give a false reading which could impact the accuracy of the results of the PCD 100. As used herein, the term “resistance” refers to the amount of time or dose it takes to achieve a change in the condition of the CI 112 or the BI 114. This resistance may be impacted by the flow resistance of the first and second compartments 108 108, 110.

Moreover, a first flow path 122 may be placed within and extend from the first opening 116 and a second flow path 124 may be placed within and extend from the second opening 118 to further control the sterilant entering the first compartment 108 and the second compartment 110, respectively. The first and second flow paths 122, 124 may be tubes, ducts, pipes, tunnels, cannulas, or any other feature which permits a flow path therethrough. As a consequence of providing the first and second flow paths 122, 124, the first flow path 122 and the second flow path 124 may each provide a pathway to the first compartment 108 and the second compartment 110, respectively. Specific to the second flow path 124, the second flow path 124 may be placed at a location relative to the BI 114 such that the ingress and egress of sterilant with respect to the BI 114 is more controlled. Specifically, the second flow path 124 may extend from the second opening 118 to a region of entry and exit with the BI 114 such that a sterilant may more readily travel into and out of the BI 114 when passing through the second flow path 124. Thus, by providing the flow paths 122, 124 the sterilizing resistance conditions within the first compartment 108 and the second compartment 110 may be controlled to a greater degree for the sterilization process by providing better control over the flow resistance to the first and second compartments 108, 110.

For example, if the CI 112 is provided in the first compartment 108, the first flow path 122 may have a specific length L1 that helps control the entry of sterilant to or sterilizing conditions within the first compartment 108. Specifically, the length L1 may range from about 0.1 millimeters (mm) to about 200 mm, such as about 0.5 mm to about 150 mm, such as about 1 millimeter (mm) to about 100 mm. Further, like the first flow path 122, the second flow path 124 may have a specific length L2 if the BI 114 is provided within the second compartment 110. Specifically, the length L2 may range from about 0.05 mm to about 100 mm, such as about 0.25 mm to about 25 mm, such as about 0.5 mm to about 50 mm. By providing the first flow path 122 with the length L1 and the second flow path 124 with the length L2, the sterilizing resistance conditions within the first compartment 108 and the second compartment 110 may be controlled by providing better control over the flow resistance to the first and second compartments 108, 110.

Further, like the lengths, L1 and L2, the first flow path 122 and the second flow path 124 may have specified widths or diameters. For example, if the CI 112 is provided in the first compartment 108, the first flow path 122 may have a specified width W1 or internal diameter that helps control the entry of sterilant or sterilizing conditions formed within the first compartment 108. Specifically, the width W1 or internal diameter may range from about 0.2 mm to about 2 mm, such as about 0.3 mm to about 1.9 mm, such as about 0.4 mm to about 1.75 mm.

Further, like the first flow path 122, the second flow path 124 may have a specific width W2 if the BI 114 is provided within the second compartment 110. Specifically, the width W2 may range from about 0.2 mm to about 2 mm, such as about 0.3 mm to about 1.9 mm, such as about 0.4 mm to about 1.75 mm. By providing the first flow path 122 with the width W1 and the second flow path 124 with the width W2, better control of the flow resistance to the first and second compartments 108, 110 enabling better control of the sterilizing conditions within the first compartment 108 and the second compartment 110 may be accomplished.

The entry of sterilant or the sterilizing resistance conditions formed within the first compartment 108 and the second compartment 110 may be further controlled by sizing the total volume V1 of the first compartment 108 and the total volume V2 of the second compartment 110 to specific dimensions in addition to making the total volumes V1, V2 of the first and second compartments 108, 110 larger than the volumes V3, V4 of the indicators 112, 114. Specifically, the total volume V1 of the first compartment 108 and the volume V3 of the CI 112 may share a proportional relationship with one another such that a certain percentage of the total volume V1 of the first compartment is free volume (i.e., volume not occupied by the CI 112). More specifically, the free volume of the first compartment 108 may range from about 30% to about 99%, such as about 50% to about 98%, such as about 85% to about 97%. The free volume of the first compartment 108 may also be an absolute value. For example, the free volume of the first compartment 108 may range from about 3 cubic centimeters (cm³) to about 30 cm³, such as about 7 cm³ to about 20 cm³, such as from about 10 cm³ to about 18 cm³. The total volume V1 of the first compartment 108 may also be defined as having a range from about 3 cm³ to about 35 cm³, such as about 7 cm³ to about 25 cm³, such as 10 cm³ to about 20 cm³.

As stated above, the entry of sterilant or the sterilizing conditions formed within the second compartment 110 may be controlled by sizing the total volume V2 of the second compartment 110 as well. Specifically, the total volume V2 of the second compartment 110 and the volume V4 of the BI 114 may share a proportional relationship with one another such that a certain percentage of the total volume V2 of the second compartment 110 is free volume. More specifically, the free volume of the second compartment 110 may range from about 30% to about 95%, such as about 50% to about 93%, such as about 85% to about 90%. The free volume of the second compartment 110 may also be an absolute value. For example, the free volume of the second compartment 110 may range from about 5 cm³ to about 35 cm³, such as about 10 cm³ to about 25 cm³, such as from about 15 cm³ to about 20 cm³. The total volume V2 of the second compartment 110 may also be defined as having a range from about 5 cm³ to about 40 cm³, such as about 10 cm³ to about 30 cm³, such as 15 cubic centimeters (cm³) to about 25 cm³.

Referring now to FIGS. 3a-3b, front and rear views of the integrated process challenge device of FIGS. 1-2 are provided according to the present disclosure. Specifically, the CI 112 and the BI 114 are shown as being placed within the first compartment 108 and the second compartment 110 of the PCD 100, respectively. Further, as shown, the first compartment 108 and the second compartment 110 may be modified such that the CI 112 and the BI 114 may be securely fit within the PCD 100. For example, the first compartment 108 may include internal posts 111 that the BI 114 may be placed, located, or positioned between. The second compartment 110 may have a specifically sized gap 117 that the BI 114 snugly fits within.

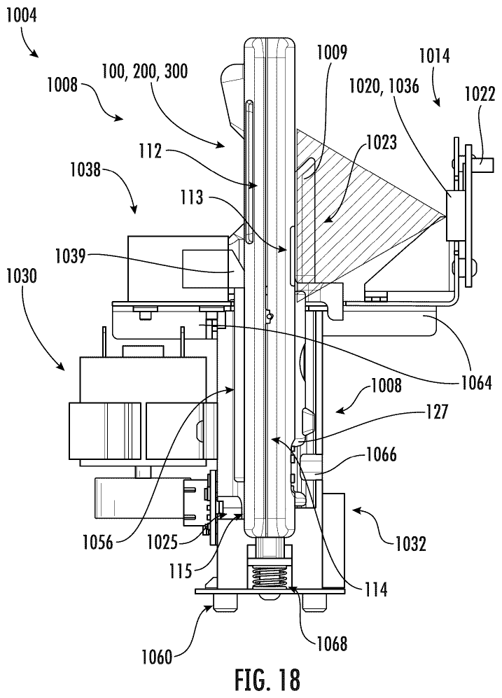

In addition, as shown, the shell 102 may have a first window region 113 and a second window region 115 such that specific regions of the PCD 100 are viewable by either a user or a digital autoreader. (See FIG. 18). By having a first window region 113 or a second window region 115, the view within the PCD 100 may be limited to the regions relevant to the verification of the effectiveness of a sterilization process using either a CI 112 or a BI 114. Alternatively, the entire PCD 100 may be composed of a transparent or translucent material such that the entirety of the contents of the PCD 100 are viewable by a user or a digital autoreader.

Referring specifically to FIG. 3a, a coding label 156 and an external chemical indicator label 158 may be placed on the PCD 100. It should be understood, however, that although the labels 156, 158 are illustrated as placed on one side of the integrated PCD 100, the labels 156, 158 may be placed on any particular side or portion of the PCD 100 as needed. The coding label 156 may contain information relevant to the specific sterilization testing the PCD 100 is intended to verify or validate. This information may be readily readable by a user or readable upon being scanned by a user by a digital autoreader as will be discussed in greater detail below. The chemical indicator label 158 may help inform a user whether the PCD 100 has already undergone exposure to the sterilization process ahead of the sterilization testing or any other indicator useful within the setting of sterilization testing utilizing PCDs such as the kind of chemical indicator as described hereinbelow.

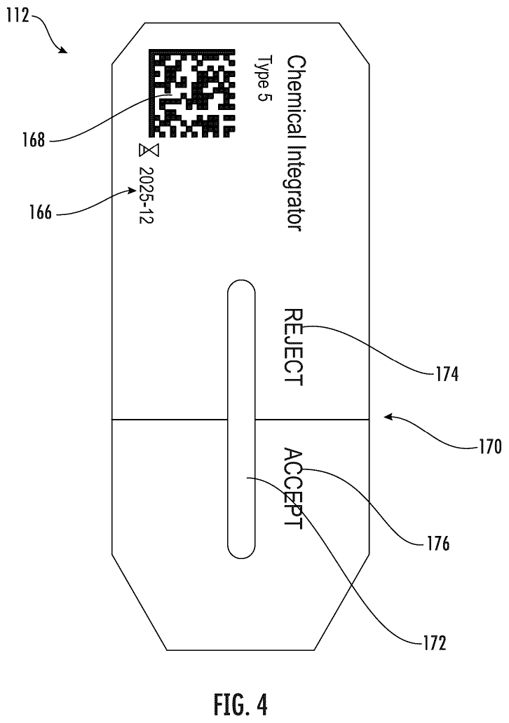

Referring now to FIG. 4, a front view of an example embodiment of an internal chemical indicator is provided according to the present disclosure. The CI 112 shown is a Type 5, pass/fail chemical indicator. As defined within the standard, ISO 11140-1, a Type 5 chemical indicator is an integrating indicator that shall be designed to react to all critical process variables to indicate exposure to a sterilization process at Stated Values (SVs). The SVs are generated to be equivalent to, or exceed, the performance requirements given in the ISO 11138-series for biological indicators. The minimum SVs shall be related to the minimum values required to achieve sterilization as specified in International Standards ISO11135, ISO 11137 (all parts), ISO 17665 (all parts), or by local regulatory agencies (see Clauses 11 and 12 of ISO 11140-1). However, it should be understood that the present disclosure is not limited to only one kind of chemical indicator. Any number of kinds of chemical indicators may be used instead of the CI 112 depicted. For example, the internal chemical indicator may be a Type 4 or Type 6 chemical indicator instead of a Type 5 chemical indicator. According to the standard, ISO 11140-1, a Type 4 chemical indicator is a multivariable process indicator that shall be designed to react to two or more of the critical process variables and is intended to indicate exposure to a sterilization process at SVs of the chosen critical process variables. A Type 6 chemical indicator is an emulating indicator that shall be designed to react to all critical process variables for specified sterilization processes. In addition, to the Type 5, Type 4, or Type 6 indicators, the CI 112 may also be a color change chemical indicator or a stationary dot chemical indicator as well. However, each of these types of chemical indicators may share commonality with the CI 112 depicted.

Thus, utilizing the currently depicted CI 112 as an example embodiment, the CI 112 may include a section of basic testing information 166 such as a lot number, an expiration date or date of use. In an exemplary embodiment, the CI 112 may also include a code 168 such as a barcode or a quick response (QR) code. The code 168 may also be a radio-frequency identification (RFID) label, a Near-field communication (NFC) tag, or other forms of scannable codes. The CI 112 may include a trackable region 170 that changes to indicate whether the prescribed sterilizing conditions have been met in the PCD 100. If the CI 112 is a pass/fail indicator, the trackable region 170 may include a travelling front 172, a fail region 174, and a pass region 176. Thus, if the travelling front 172 visually advances to the pass region 176, the CI 112 will indicate that the necessary sterilizing conditions have been met to sterilize the PCD 100. Conversely, if the travelling front 172 remains in the fail region 174, the CI 112 will indicate that the necessary sterilizing conditions have not been met to sterilize the PCD 100.

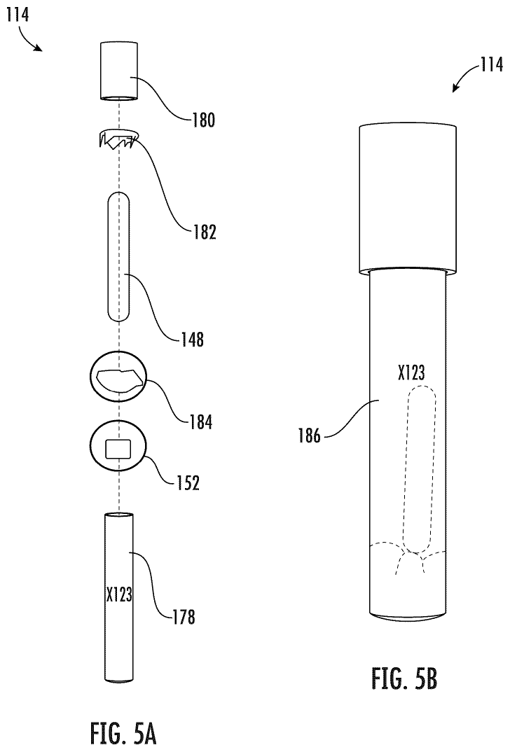

Referring now to FIGS. 5a-5b, various views of an example embodiment of a biological indicator are depicted according to the present disclosure. The BI 114 shown is a self-contained biological indicator (SCBI). However, like the CI 112, it should be understood that the present disclosure is not limited to only one type of biological indicator. Any number of types of biological indicators may be used instead of the BI 114 depicted. For example, the biological indicator may be a self-contained biological indicator where germination/outgrowth of spore or growth of the microorganism may detect by a detection method such as but not limited to: a color change or turbidity of the growth medium, production of fluorescent compounds within the biological indicator, a change in conductivity of the biological indicator, or of a specific gas or volatile compound produced by the biological indicator. However, each of these types of biological indicators share a functional commonality with the BI 114 depicted, i.e., a response to growth conditions if not rendered sterile or the absence of this response if rendered sterile.

Thus, utilizing the currently depicted BI 114 as an example embodiment, the BI 114 may include a container of growth medium 148 (such as a glass vial) and a biological culture sample 152. The biological culture sample 152 may include a culture or colony of viable microorganisms capable of providing a specified resistance (i.e., resistance associated with duration of exposure to sterilizing conditions or to an integrated dose of sterilant) to a specified sterilization process. For example, microbial species and strains are selected as biological indicator candidates based on their known resistance to the specific method of sterilization. For steam sterilization, moist heat sterilization, or hydrogen peroxide sterilization processes, the test organisms employed can be spores of a suitable strain of bacteria such as a strain of Geobacillus stearothermophilus. For example, test organisms employed for an ethylene oxide sterilization processes can be spores of a suitable strain of Bacillus subtilis or Bacillus atrophaeus, as noted in ISO 11138-part 2. The resistance of the test organism is critical for verifying or validating sterilization and should be known and determined as appropriate. The container of growth medium 148 is provided to support the growth of the microorganisms contained within the biological culture sample 152.

Beyond the biological culture sample 152 and the container of growth medium 148, the BI 114 may also include other components such as a container 178 to house the components of the BI 114, a cap 180 that connects with the container 178, a cap filter 182 that prevents the ingress of undesired contaminants, a support 184 for the biological culture sample 152, and a label 186 which can provide information relevant to the BI 114 or the PCD 100.

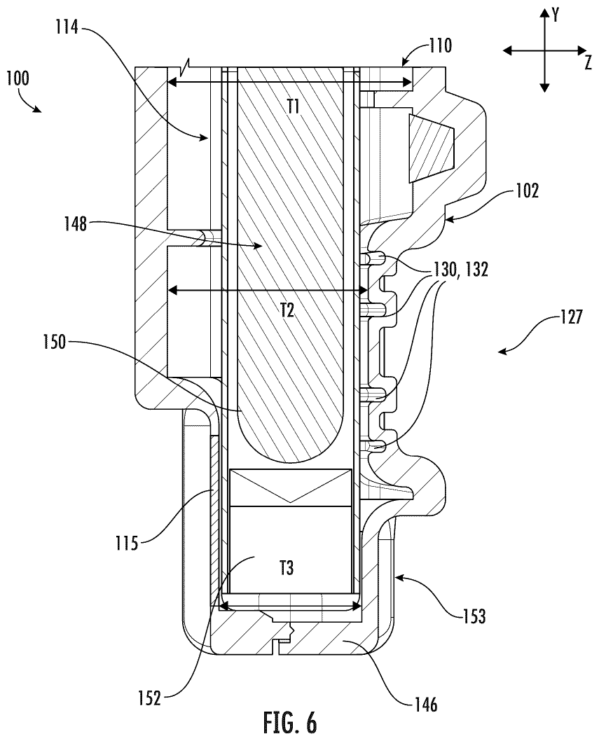

Referring now to FIG. 6, a partial side cutaway view of an embodiment of an integrated process challenge device is provided according to the present disclosure, particularly illustrating the BI 114 placed within the integrated process challenge device 100. As shown, the BI 114 is placed within the second compartment 110 at an end 146 of the PCD 100. The PCD 100 and the second compartment 110 may be designed in such a manner that all of the processing and testing of the BI 114 contained therein may be conducted without opening the PCD 100. Specifically, the PCD 100 may include a compressible section 127 defined along the second compartment 110. The compressible section 127 may allow for the localized compression of the PCD 100 such that the container of growth medium 148 within the BI 114 may be shattered and the contents contacted with the biological culture sample 152. To achieve this, the compressible section 127 may include a variety of features. For example, the compressible section 127 may include a plurality of ribs 130 protruding outward from the shell 102. Each of the plurality of ribs 130 may include a hollow interior 132 which allow for flexion and compression when pressure is applied to the compressible section 127.

The compressibility of the compressible section 127 may also be enhanced or alternatively achieved by providing a variety of thicknesses relative to the maximum thickness T1 of the PCD 100. Specifically, the variable thickness may be smaller around a portion of the compartment of the BI 114 than other portions of the shell 102. For example, a thickness T2 around the compressible section 127 may be smaller than the maximum thickness T1 of the PCD 100. The thickness T2 may also taper inward to a thickness T3 of the end 146 of the PCD 100. By providing these varying thicknesses T1, T2, T3, an end 150 of the container of growth medium 148 may be pressed against the shell 102 such that the end 150 of the container of growth medium 148 fractures.

The reduced thickness T3 of the end 146 may also provide other benefits. For example, if heat is being provided to incubate the biological culture sample 152 with the growth media contained within the container of growth medium 148, the reduced thickness T3 may allow for heat to penetrate through the shell more readily 102. Easier thermal penetration may allow for the microorganisms contained with the biological culture sample 152 to be incubated at a desired temperature more easily without removing the biological culture sample 152 from the PCD 100. Thus, by providing the reduced thickness T3 of the shell 102, a thermal region 153 may be created along the shell 102.

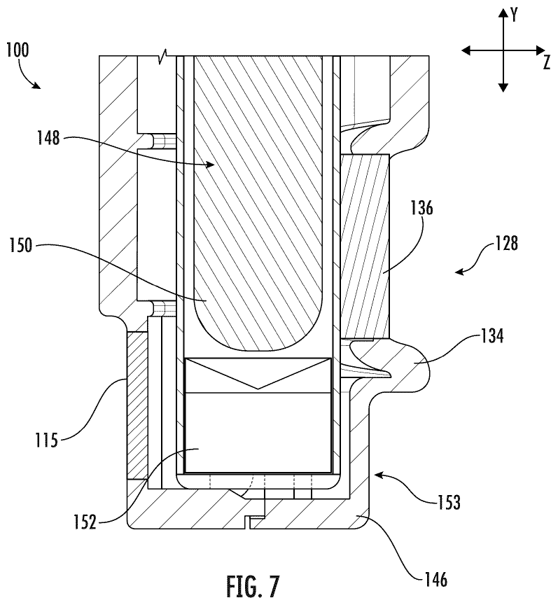

Referring now to FIG. 7, a partial side cutaway view of another embodiment of a biological indicator placed within an integrated process challenge device is provided according to the present disclosure. As shown, a frangible section 128 may be provided instead of the compressible section 127. The frangible section 128 may be a different material 136 from the material 134 of the shell 102. Specifically, the material 136 may be a foil or another readily frangible material. By providing the different material 136, the frangible section 128 may more readily compress toward the container of growth medium 148 and result in the fracturing of the container of growth medium 148.

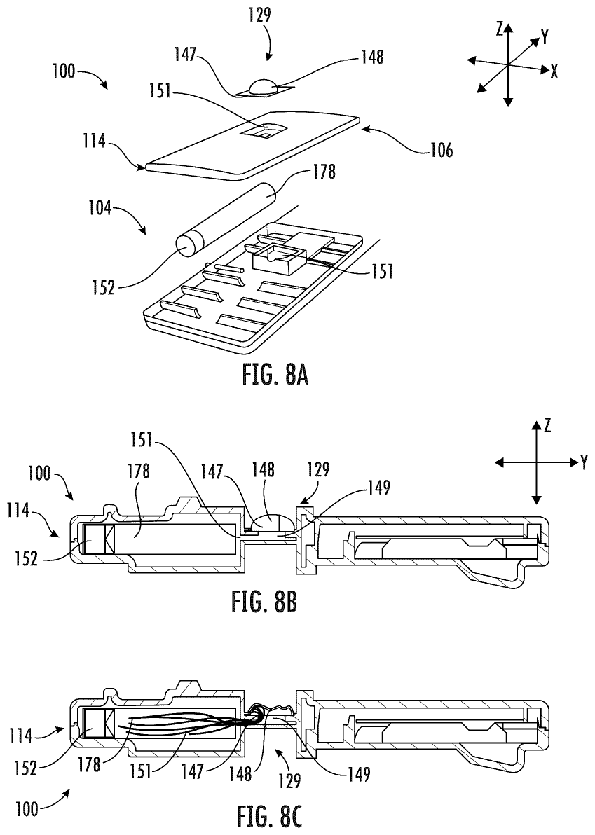

Referring now to FIGS. 8a-8c, various views of an embodiment of a compressible region of an integrated process challenge device 100 are provided according to the present disclosure. In particular, FIGS. 8a-8b provide views of a compressible region, while FIG. 8c provides a view of the compressible region being ruptured inwardly. As shown, the compressible region 129 of the PCD 100 may have a different configuration than the compressible section 127 or the frangible section 128 discussed with reference to FIGS. 6-7. Specifically, the compressible region 129 may include the container of growth medium 148 placed in a compressible material such as a semi-rigid or semi-flexible material that is capable of undergoing the conditions associated with sterilization. The container of growth medium 148 may also be loosely sealed with a frangible portion 147 that acts as a path of least resistance for the container of growth medium 148 if pressure is applied. For example, the frangible portion 147 may be a scored region or a thinner piece of material than the other portions of the container of growth medium 148. The frangible portion 147 may also be a material such as a foil or another readily frangible material. The compressible region 129 may also include a window 149 in which the frangible portion 147 is placed on the window 149. The window 149 provides an entry point to the interior 104 of the PCD 100.

As a consequence of providing the frangible portion 147 and the window 149, applying pressure to the container of growth medium 148 will result in the contents of the container 148 travelling into the interior 104 of the PCD 100 through the frangible portion 147 and the window 149. Further, to assist the flow of the contents of the container of growth medium 148, a funnel 151 may be provided in the interior 104 of the PCD 100. Thus, when the compressible region 129 described in FIGS. 8a-8b is utilized, a force may be applied to the container of growth medium 148 which results in the frangible portion 147 rupturing toward the interior 104 of the PCD 100 as shown in FIG. 8c. Then, the contents of the container of growth medium 148 may travel through the window 149 and be directed toward the container 178 of the BI 114 through the funnel 151 resulting in the contents of the container of growth medium 148 being incorporated with the biological culture sample 152 placed within the container 178 of the BI 114. By providing this configuration, the contents of the container of growth medium 148 may be mixed with the biological culture sample 152 without opening the PCD 100.

Regardless of the features of the compressible section 127, the frangible section 128, or the compressible region 129, the container of growth medium 148 may be oriented within or on the shell 102 such that the growth media contained within the container 148 can be substantially incorporated with the biological culture sample 152. For example, referring to FIGS. 6-7, the end 150 of the container 148 may be aligned with the compressible section 127 or the frangible section 128 (at the lower end 150 of the container 148) such that the container 148 can rupture toward the biological culture sample 152. By providing this orientation, any fragments of the container 148 which could act as a cup for the growth media and prevent the growth media from being contacted with the biological culture sample 152 may be reduced. Alternatively, the compressible region 129 which includes the container of growth medium 148 may be provided on the exterior 106 of the shell 102 such that the container 148 is incapable of preventing the growth media from contacting the biological culture sample 152.

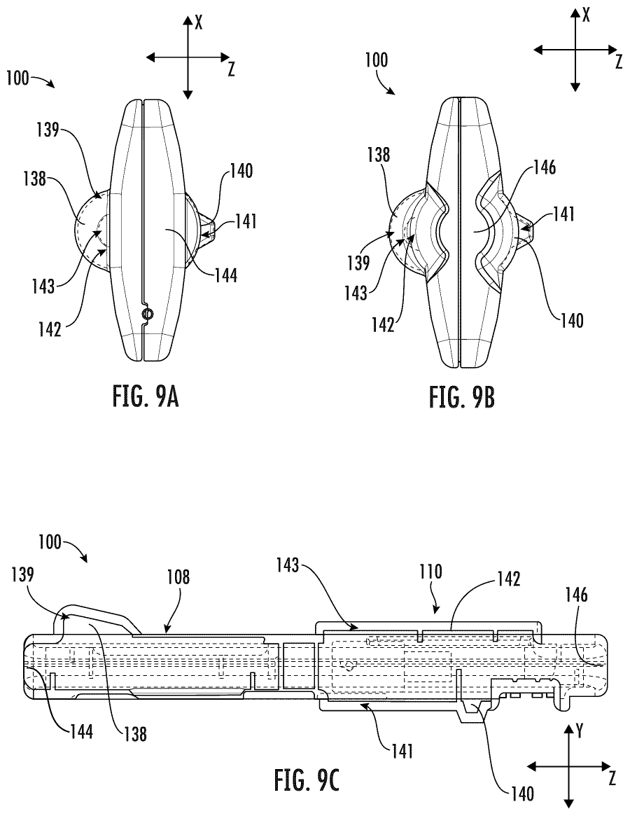

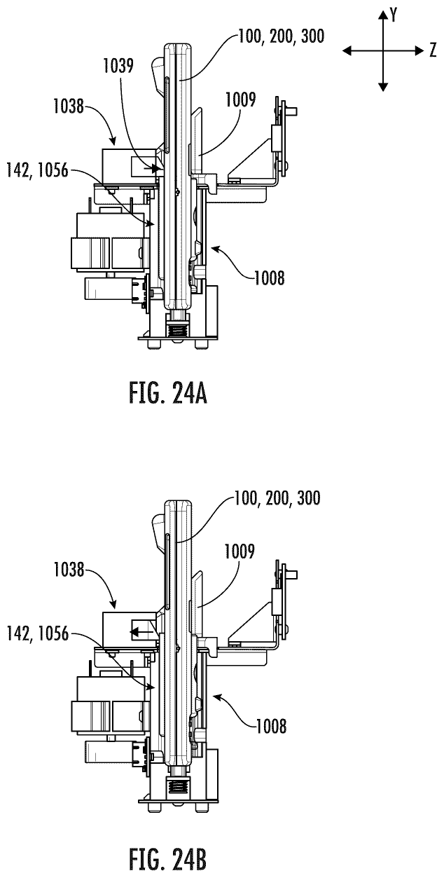

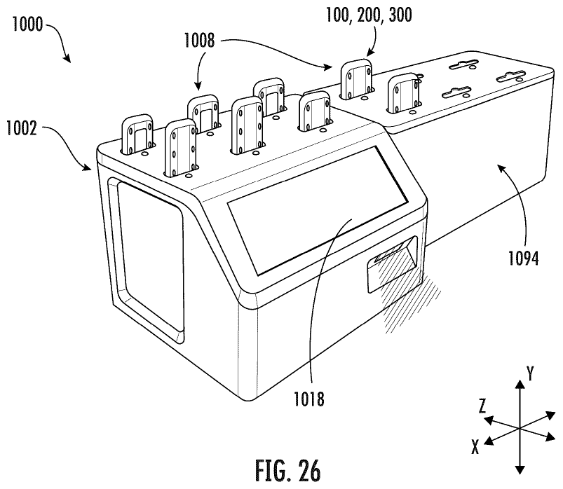

Referring now to FIGS. 9a-9c, various views of an embodiment of an integrated process challenge device are provided according to the present disclosure. Specifically, FIG. 9a provides a top view, FIG. 9b provides a bottom view, and FIG. 9c provides a side view. As shown, the integrated PCD 100 may include a variety of locating features 138, 140, 142. Specifically, a first locating feature 138 may be provided toward a first end 144 around the first compartment 108 of the PCD 100. A second locating feature 140 may be provided toward a second end 146 of the PCD 100. A third locating feature 142 may also be provided toward the second end 146 of the PCD 100 opposite the second locating feature 140. As depicted, each of the locating features 138, 140, 142 may be protrusions on the shell 102. However, it should be understood that the locating features 138, 140, 142 may be recesses, shavings, or other forms of locating features. Each of the locating features 138, 140, 142 may help properly or correctly orient the PCD 100 within a digital autoreader as will be discussed in greater detail below. (See FIG. 24a-24b and 26).

In addition, the locating features 138, 140, 142 may help set an internal volume within the first compartment 108 and the second compartment 110. Specifically, the first locating feature 138 may include a hollow interior 139; the second locating feature 140 may include a hollow interior 141; and the third locating feature 142 may include a hollow interior 143. As a result of providing the hollow interiors 139, 141, 143, the total interior volume of the first compartment 108 and the second compartment 110 may be expanded. Thus, by augmenting the interior volume, the hollow interiors 139, 141, 143 may help determine the rate at which sterilant enters and exits from the first compartment and the second compartment as discussed above. As discussed above, the locating features 138, 140, 142 may also be recesses. If the locating features 138, 140, 142, the locating features 138, 140, 142 may reduce the total interior volume of the first compartment 108 and the second compartment.

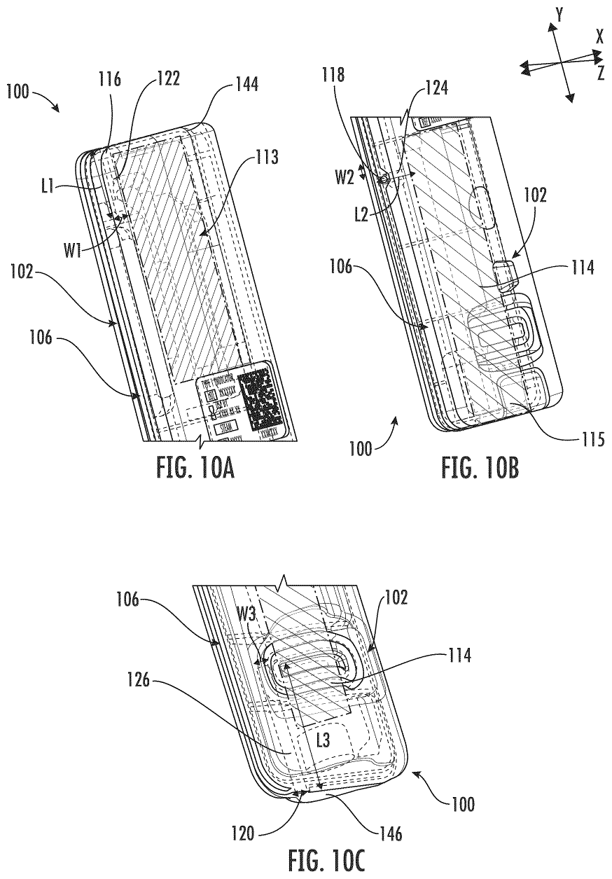

Referring now to FIGS. 10a-10c, various views of embodiments of an integrated process challenge device are provided according to the present disclosure, particularly illustrating flow paths capable of facilitating flow of a sterilant into and from the interior of the integrated process challenge device. As shown, the first flow path 122 is shown as extending in the linear Y-direction, and the second flow path 124 is shown as extending in the X-direction. However, if the first and second openings 116, 118 are provided in different locations on the shell 102, the flow paths 122, 124 may be provided in other directions or multiple directions as will be discussed below. In addition, as shown, a third opening 120 may be provided on the shell 102. Like the first and second opening 116, 118, the third opening 120 may be sized such that a sterilant may enter the PCD 100 through the third opening 120. Further, like the first and second openings 116, 118, the third opening 120 may include a third flow path 126 extending from the third opening 120 and providing a pathway to the interior 104 of the shell 102. Further, like the first and second flow paths 122, 124, the third flow path 126 may be a tube, duct, pipe, tunnel, cannula, or any other feature which permits a flow path therethrough. Moreover, like the first and second flow paths 122, 124, the third flow path 126 may provide a pathway to the second compartment 110. Further, like the first and second flow paths 122, 124, the third flow path 126 may help control the entry of sterilant or sterilizing conditions formed within the second compartment 110. As shown, the third opening 120 and the third flow path 126 may be provided instead of the second opening 118 and the second flow path 124. However, the third opening 120 and the third flow path 126 may be provided in addition to the second opening 118 and the second flow path 124.

Specifically, if the third opening 120 and the third flow path 126 are provided, the third flow path 126 may have a specific length L3 that helps control the entry of sterilant or sterilizing conditions formed within the second compartment 110. Specifically, the length L3 may range from about 0.2 millimeters (mm) to about 400 mm, such as about 1 mm to about 300 mm, such as about 2 millimeter (mm) to about 200 mm. In addition, the length L3 may be defined such that the third flow path 126 extends from the third opening 120 to a region of entry and exit within the BI 114. By defining the third flow path 126 as having such a length L3, the third flow path 126 may better control the ingress and egress of sterilant to and from the BI 114. Further, like the first flow path 122 and the second flow path 124, the third flow path 126 may have specified widths or diameters. For example, if the BI 114 is provided in the second compartment 110, the third flow path 126 may have a specified width W3 that helps control the entry of sterilant or sterilizing conditions formed within the first compartment 108. Specifically, the width W3 may range from about 0.2 mm to about 2 mm, such as about 0.3 mm to about 1.9 mm, such as about 0.4 mm to about 1.75 mm. By providing the third flow path 126 with the width W3, the sterilizing conditions within the second compartment 110 may be further controlled.

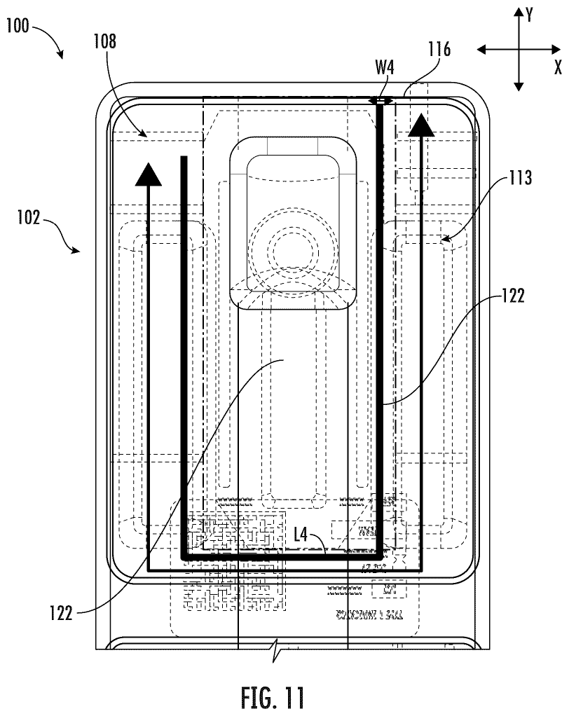

Referring now to FIG. 11, a back, cutaway view of an embodiment of an integrated process challenge device is provided. As shown, a baffle 155 may be provided within the first compartment 108 to direct the control the flow of sterilant within the first compartment 108. In particular, the baffle 155 may prevent sterilant from reaching the CI 112 directly or in a linear manner. For example, the baffle 155 may be a multidirectional baffle such that an internal multidirectional pathway is formed within the first compartment 108. However, although the baffle 155 is described as being used with respect to the first compartment 108, the baffle 155 may also be provided in the second compartment 110 if desired.

Still referring to FIG. 11, the baffle 155 may travel in a hook shaped or u-shaped manner. Specifically, the baffle 155 may begin travelling downward in the Y-direction, then travel in the X-direction, then travel upward in the Y-direction. In addition, if the baffle 155 has an internal multidirectional pathway, baffle 155 may have a length L4. Specifically, the length L4 may range from about 70 mm to about 270 mm, such as about 90 mm to about 200, such as about 110 mm to about 150 mm. Further, the baffle 155 may have a width W4. In particular, the width W4 may range from about 0.5 mm to about 2 mm, such as about 0.75 mm to about 1.5 mm, such as about 0.9 mm to about 1.2 mm. Like the other lengths and widths discussed, the length L4 and W4 may help control the sterilizing conditions or amount of sterilant that enters or exits the interior 104 of the PCD 100. However, when an internal multidirectional pathway is provided via the baffle 155, the conditions of the sterilization process may be more controlled within the PCD 100. Specifically, because the internal multidirectional pathway can extend the distance sterilant must travel to reach the CI 112 or BI 114, the resistance of the PCD 100 for the CI 112 or BI 114 or the integrated sterilant dwell time with the CI 112 or BI 114 may be controlled which may result in greater resistance specific to the sterilization process. As a result of providing the baffle 155 as described herein, the resistance of the first compartment 108 or the second compartment 110 may be better controlled by controlling the flow resistance within the first and second compartments 108, 110.

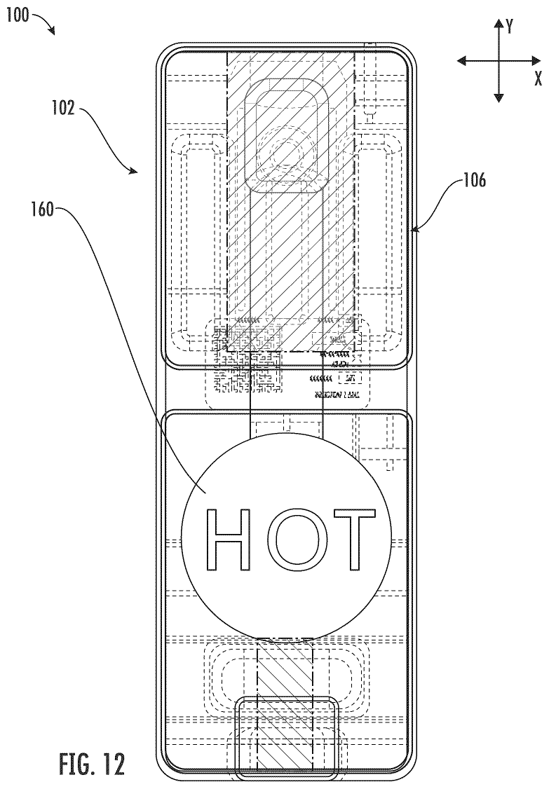

Referring now to FIG. 12, a rear view of an embodiment of an integrated process challenge device with a thermal indicating label is illustrated according to the present disclosure. As shown, the PCD 100 may include a thermal indicating label 160 placed on the exterior 106 of the shell 102 of the PCD 100. The thermal indicating label 160 may provide a visual representation of whether the PCD 100 is at a safe temperature to handle after undergoing the desired sterilizing conditions. Specifically, the thermal indicating label 160 may have a changing visual indication that changes based on the temperature of the PCD 100. For example, the thermal indicating label 160 may change in color or appearance from a pre-sterilized state to a post-sterilized state based on the temperature of the PCD 100 and thus indicating whether the PCD 100 has been subjected to sterilizing conditions.

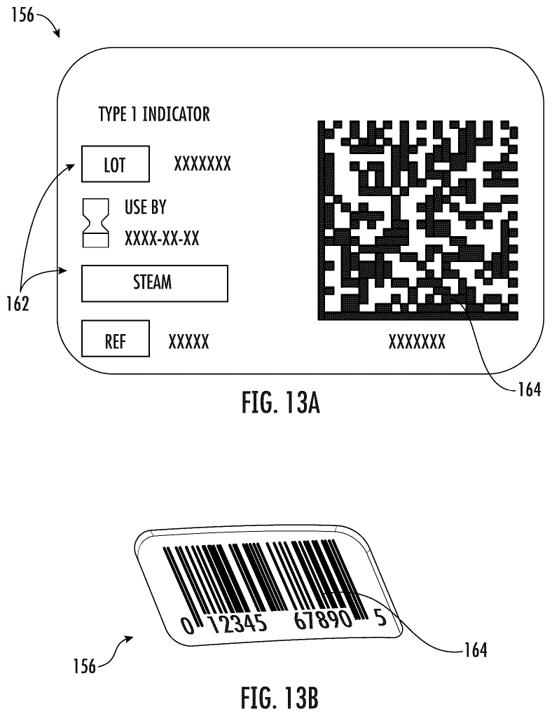

Referring now to FIGS. 13a-13b, various example embodiments of codes that can be applied to an integrated process challenge device are illustrated according to the present disclosure. As shown, the coding label 156 may include information 162 relevant to the particular test of the PCD 100 such as the type of sterilization being utilized such as steam or thermal (e.g. low temperature), the product information (e.g. unique device identifier and lot) of the PCD 100, expiration date information, or reference numerals particular to the PCD 100. The coding label may also include a code 164 that provides information when scanned. In exemplary embodiments, the code 164 may be a barcode (as shown in FIG. 13b) or a QR code (as shown in FIG. 13a). The code 164 may also be an RFID label, an NFC tag, or other forms of scannable codes. The scanning of the information can also provide traceability of the results of the test of the PCD 100.

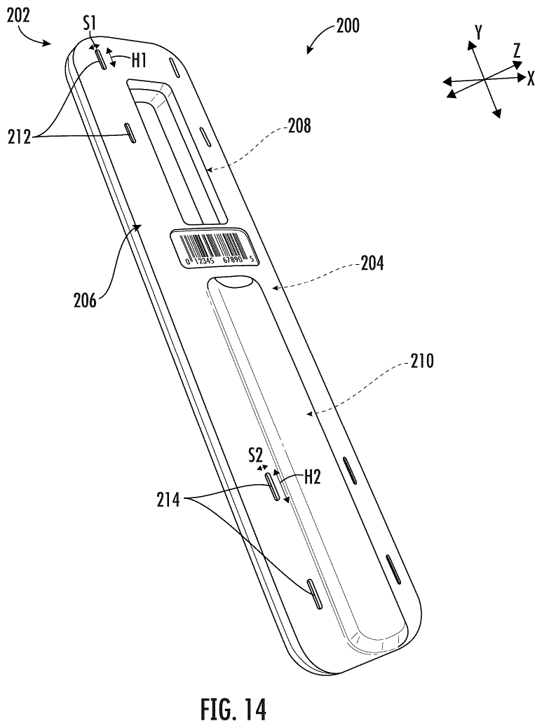

Referring now to FIG. 14, a perspective view of an embodiment of an integrated process challenge device is provided according to the present disclosure, particularly illustrating ports capable of facilitating flow of a sterilant into and from the interior of the integrated process challenge device. As shown, the PCD 200 includes a shell 202 having an interior 204 and an exterior 206. The shell 102 also provides for a first compartment 208 and a second compartment 210 in the interior of the shell 202. Further, as shown, the PCD 200 may include a plurality of ports 212 along the first compartment 208. The PCD 200 may also include a plurality of ports 214 on the second compartment 210. Each of the plurality of ports 212, 214 may allow for the ingress of sterilant or formation of sterilizing conditions within either the first compartment 208 or the second compartment 210. Further, the amount of sterilant or sterilizing conditions formed within either the first compartment 208 or the second compartment 210 may be controlled by selecting a specific number of ports and/or a specific size for the individual ports of either the first compartment 208 or the second compartment 210. For example, 2 to 8 ports may be provided along each compartment 208, 210 of the PCD 200.

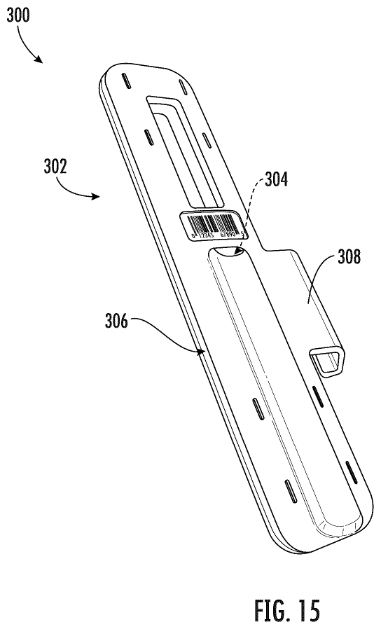

Referring now to FIG. 15, a perspective view of an embodiment of an integrated process challenge device 300 is shown according to the present disclosure, particularly illustrating a support formed with the integrated process challenge device 300. As shown, the PCD 300 includes a shell 302 having an interior 304 and an exterior 306. On the exterior 306 of the shell 302, a support 308 may be provided. For example, the support 308 may take the form of a hook or a clip provided on the exterior of the shell 302 to enable the PCD 300 to be attached to a load location or to a location on or within a sterilizer cart. In some aspects of the invention, it may be beneficial to attach the PCD 300 using the support 308 to a location within a sterilizer that is known or suspected to be the most difficult location to sterilize within the sterilizer.

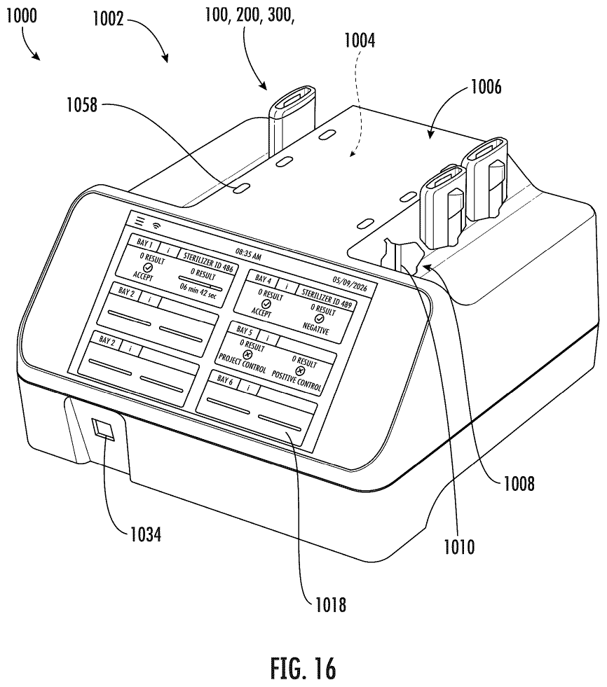

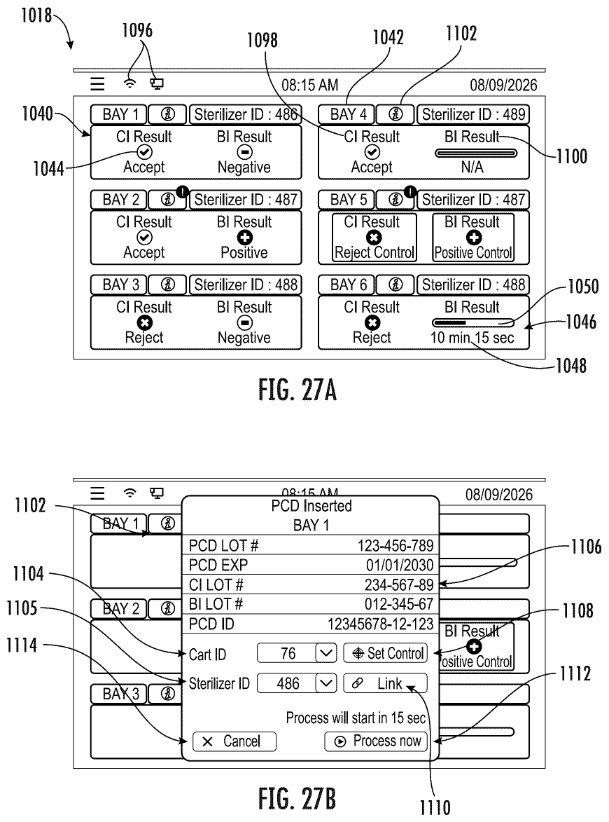

Referring now to FIG. 16, a perspective view of an embodiment of a digital autoreader is illustrated according to the present disclosure. As shown, the digital autoreader 1000 includes a housing 1002 having an interior 1004 and the exterior 1006. The digital autoreader 1000 also includes a well 1008 or plurality of wells 1008 with an opening 1010 for receiving a process challenge device, such as the PCD 100. However, it should be understood that although the digital autoreader 1000 will be described as used with the PCD 100, the digital autoreader 1000 may also accommodate and be used with the PCDs 200, 300. Still referring to FIG. 16, the digital autoreader 1000 may include an interface 1018 and an exterior scanner 1034. The exterior scanner 1034 may allow for the scanning of the code 164 of the coding label 156. Once scanned, the information from the code 164 can appear on the interface 1018 as will be discussed in greater detail below. (See FIGS. 27a-27b). Further, the interface 1018 can alert a user if the PCD 100 has been successfully sterilized as will be discussed in greater detail below.

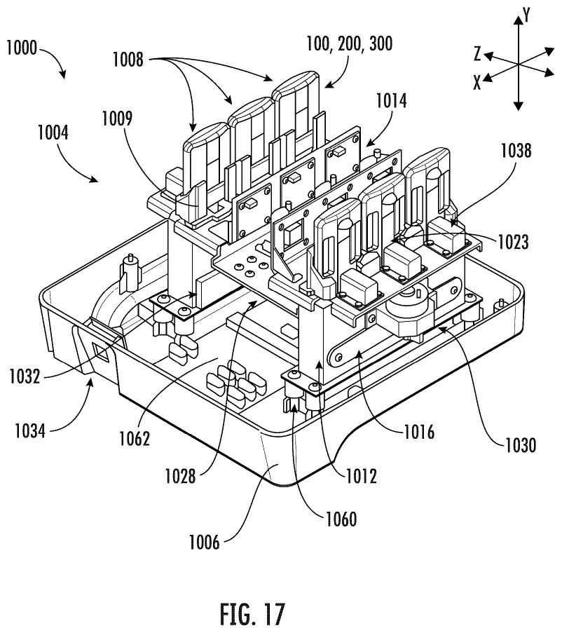

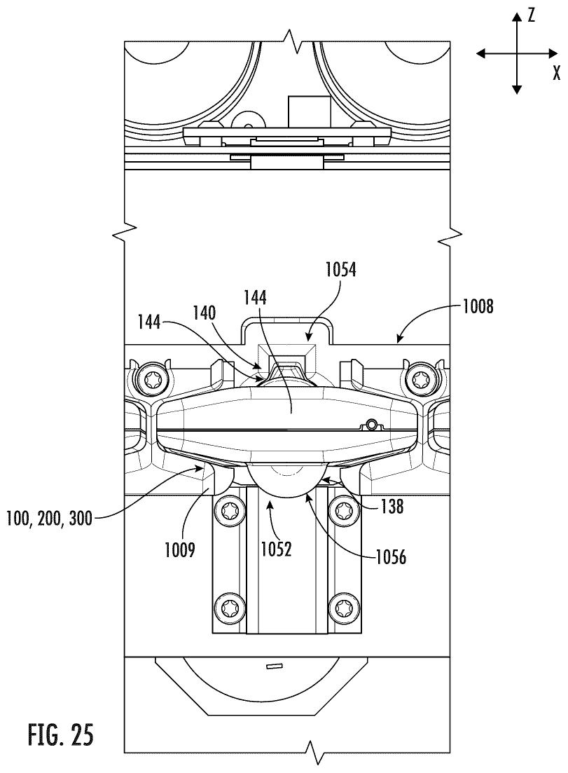

Referring now to FIG. 17, another perspective view of the digital autoreader of FIG. 16 is illustrated according to the present disclosure, particularly illustrating the interior of the digital autoreader. As shown, the interior 1004 of the digital autoreader 1000 includes the well(s) 1008. The well(s) 1008 may include an alignment strip 1009 that helps orient the PCD 100 within the well 1008 as will be discussed in greater detail with reference to FIGS. 24a-24b and FIG. 25. The well(s) 1008 may also include a block 1012 for retaining the PCD 100. The interior 1004 of the digital autoreader 1000 may also include a first sensor 1014 and a second sensor 1016. The first sensor 1014 and the second sensor 1016 may be positioned on or adjacent to the block 1012 of the well(s) 1008. The digital autoreader 1000 may also include a crushing mechanism 1028, a mixing mechanism 1030, and a heating element 1032 for use with the BI 114 as will be discussed in greater detail below. The digital autoreader 1000 may also include a circuit board that contains processors in communication with the first and second sensors 1014, 1016 and the interface 1018.

Referring to FIG. 18, a side view of an embodiment of a well of a digital autoreader is shown according to the present disclosure. As shown, the first sensor 1014 may be configured to read a variable of the first indicator or the CI 112 through the first window region 113 and determine whether the PCD 100 has been adequately sterilized. Specifically, the alignment strip 1009 may include an opening 1023 that the first sensor 1014 can scan the CI 112 through. Further, the second sensor 1016 may also be configured to read a variable of the second indicator or the BI 114 through the second window region 115 and determine whether the PCD 100 has been adequately sterilized. Specifically, the well 1008 may include an opening 1025 that the second sensor 1016 can read the BI 114 through.

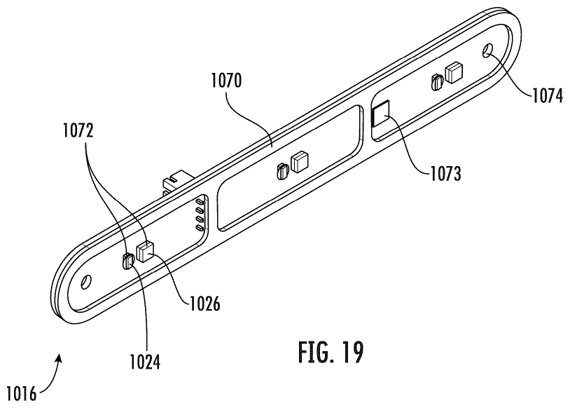

In addition, if the first indicator is a CI 112, the first sensor 1014 may be a chemical indicator reader 1014. Specifically, the first sensor 1014 may be or include a camera 1020 such as a machine vision camera. More specifically, the camera may include a processor 1022 with a machine learning algorithm programmed therein. The machine learning algorithm may then be configured to read the CI 112. For example, the machine learning algorithm may be configured to read the information of the CI 112 (i.e., the chemical indicator code 168 or the chemical testing information 162) and the trackable region 170 described above with reference to the CI 112. Thus, the machine learning algorithm may be configured to read the information of the CI 112 and determine a change in an appearance of the CI 112 corresponding to whether or not the PCD 100 has undergone conditions sufficient to sterilize the PCD 100. Moreover, if the second indicator is a BI 114, the second sensor 1016 may be a biological indicator autoreader 1016 as will be discussed in greater detail below with reference to FIG. 19.

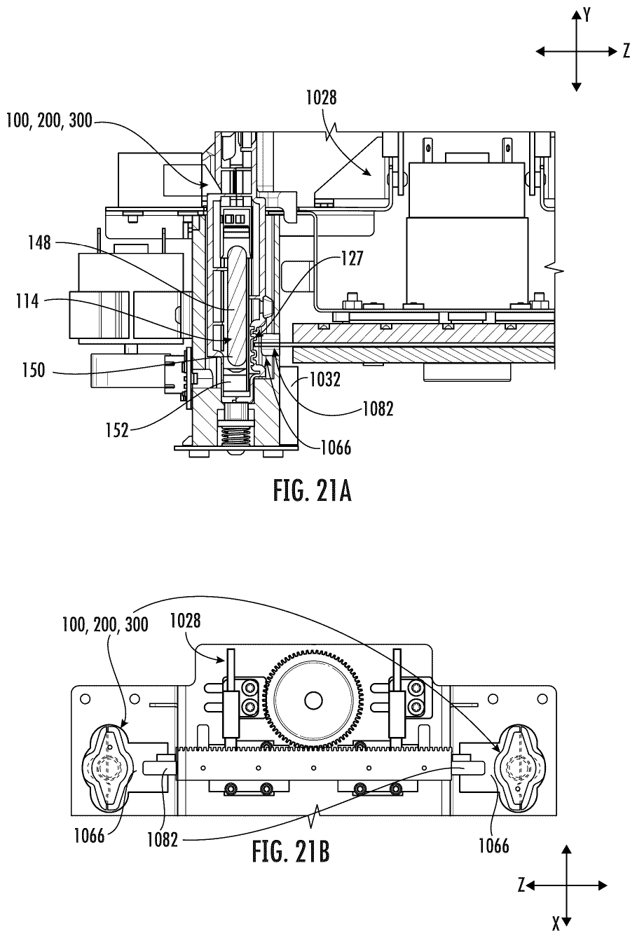

Still referring to FIG. 18, the interior 1004 of the housing 1002 may include an interior scanner 1036 that scans relevant information on the PCD 100 such as the code 164 of the coding label 156. The interior scanner 1036 may be the camera 1020. Alternatively, the interior scanner 1036 may be a separate component of the digital autoreader 1000 and operate independently from the camera 1020. However, if the interior scanner 1036 is independent from the camera 1020, the interior scanner may be a second camera, a barcode scanner, a QR code scanner, an RFID reader, an NFC reader, or any other form of scanner. The interior 1004 of the housing 1002 may also include a locking mechanism 1038 with an extendable bar 1039. Further, the block 1012 of the well(s) may include a complementary locating feature 1056 that engages with the bar 1039. The well(s) 1008 may include a support plate 1064 to support or otherwise hold up components of the well(s) 1008 or the interior 1004 of the housing 1002 such as the first sensor 1014 or the locking mechanism 1038. Specifically, the first sensor 1014 or the locking mechanism 1038 can be mounted to the support plate 1064 which is then mounted to the block 1012 of the well(s) 1008. The well(s) 1008 may also include an opening 1066 through which a crusher 1082 of the crushing mechanism 1028 can contact the compressible section 127 of the PCD 100 through the block 1012 of the well(s) 1008. (See FIG. 21a). The well(s) 1008 may also include an ejection mechanism 1068. The ejection mechanism 1068 may eject the PCD 100 or PCDs 100 from the well(s) 1008 when activated.

Referring now to FIG. 19, a perspective view of an embodiment of a biological indicator reader is illustrated according to the present disclosure. For example, the second sensor 1016 or the biological indicator reader 1016 may include a photoemitter 1024 and a photodetector 1026. A gasket 1070 may also be provided along the rim and portions of the biological indicator reader 1016 to electrically isolate the components of the biological indicator reader 1016 such as individual pairs of photoemitters 1024 and a photodetectors 1026. The gasket 1070 may also be provided to better isolate light emitted and received by a pair of a photoemitter 1024 and a photodetector 1026 corresponding to a PCD 100 from a separate pair of a photoemitter 1024 and a photodetector 1026 for a separate PCD 100.

In addition, the photoemitter 1024 may emit light or photons toward the BI 114, the light or photons will pass through the PCD 100 to the BI 114 contained within the second compartment 110 which may absorb the light or photons. The BI 114 may then emit light or photons of a similar or longer wavelength and the emitted light or photons may be captured by the photodetector 1026. Through detecting the presence of emitted light or photons, the biological indicator reader 1016 may be capable of detecting subtle changes within the BI 114 such as whether biological activity is occurring within the BI 114. Specifically, the biological indicator reader 1016 may be capable of detecting fluorescence or a fluorescent signal within the incubated biological culture sample 152 to determine if enzymatic activity is occurring. This detectable enzymatic activity would then communicate whether the biological culture sample 152 has been sterilized by the sterilizing conditions that a PCD 100 was placed under. The biological indicator reader 1016 may also be capable of determining whether growth media is properly incorporated with the biological culture sample 152 as discussed below. Specifically, the biological indicator reader 1016 can be configured to detect at least one of a volume of a growth medium contained within the container 148 or a saturation level of the biological culture sample 152 with the growth medium. To this end, the wavelength emitted and received by the photoemitter 1024 and the photodetector 1026, respectively, may be selected such that the biological indicator reader 1026 is capable of detecting the aforementioned changes within the BI 114 through the PCD 100 while not necessarily needing a complex design. As a result of selecting a particular range of wavelengths capable of penetrating through the PCD 100 into and out of the second compartment 110, the photoemitter 1024 and photodetector 1026 may be able to be placed on the same biological indicator reader 1026 and still be capable of detecting changes within the BI 114. Further, the photoemitter 1024 and the photodetector may also not need a filter to be able to adequately detect the changes within the BI 114. In addition, the BI 114 may be read without removing the BI 114 from the PCD 100.

Further, the photoemitter 1024 or photodetector 1026 may include or connect to a processor 1072 to process the information from the photoemitter 1024 and the photodetector 1026. Moreover, the processor 1072 may also include a machine learning algorithm programmed therein. The machine learning algorithm may be configured to read the BI 114 and determine the change in biological activity of the BI 114. Accordingly, the biological indicator reader 1016 may help determine whether the BI 114 has been sterilized. Furthermore, the biological indicator reader 1016, may also include a camera, barcode scanner, a QR code scanner, an RFID reader, or an NFC reader in addition to the components discussed above.

Additionally, the biological indicator reader 1016 may also include a heat sensor 1073. The heat sensor 1073 may be configured to provide a feedback loop for a heating element 1032 which will be described in greater detail hereinbelow. The biological indicator reader 1016 may also include a variety of other sensors either in addition to or instead of the photoemitter 1024 and photodetector 1026.

For example, the biological indicator reader 1016 may include a gas sensor or a conductivity sensor (not shown). A gas sensor may be capable of detecting gasses emitted by the incubated biological culture sample 152 which then could be used to determine if the biological culture sample 152 had been inactivated by the sterilizing conditions that the PCD 100 was placed under. A conductivity sensor may be able to measure the electrical conductivity of the incubated biological culture sample 152 to verify if biochemical reactions occur which would indicate whether the biological culture sample 152 has been inactivated by the sterilizing conditions that the PCD 100 was placed under.

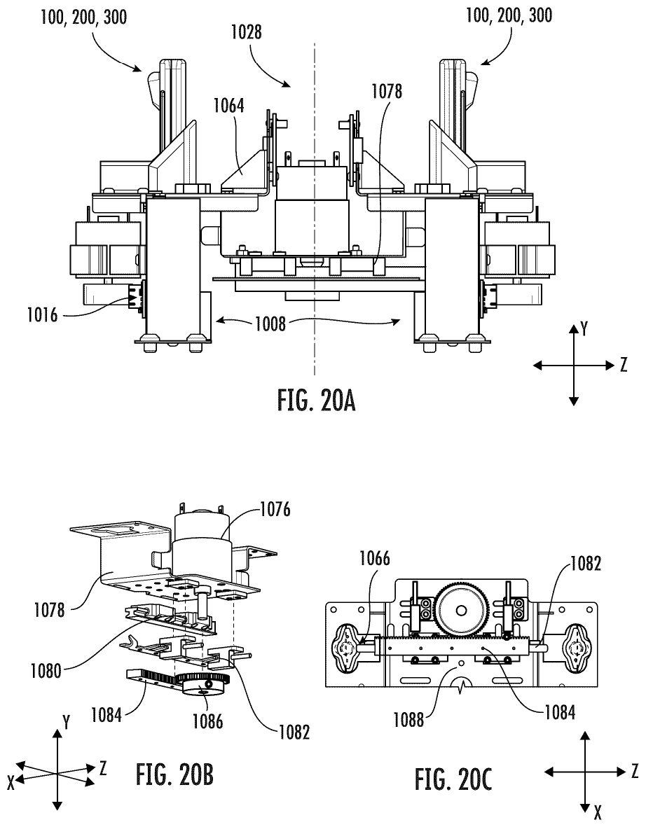

Referring now to FIGS. 20a-20c and FIGS. 21a-21b, various views of an embodiment of a crushing mechanism being used in conjunction with a process challenge device are provided according to the present disclosure. As discussed above, the crushing mechanism 1028 can contact the compressible section 127 of the PCD 100 through the opening 1066 of the block 1012. (See FIGS. 21a-21b). Specifically, the crushing mechanism 1028 can include a motor 1090 that can drive a crusher 1082 into the compressible section 127 of the PCD 100. The crushing mechanism 1076 can also include a support plate 1078 for supporting or otherwise holding up the crushing mechanism 1076. Specifically, the support plate 1078 can be attached to the support plate 1064 which is connected to the block 1012 of the well(s) 1008. Further, the crushing mechanism 1076 can include a rail 1080 to guide the crusher 1082 in a linear direction 1088 such as in the Z-direction. The crushing mechanism 1076 can also include a rack 1084 that interacts a gear 1086 driven by the motor 1090 thereby propelling the rail 1080 and the crusher 1082 toward the compressible section 127 of the PCD 100. (See FIGS. 21a-21b).

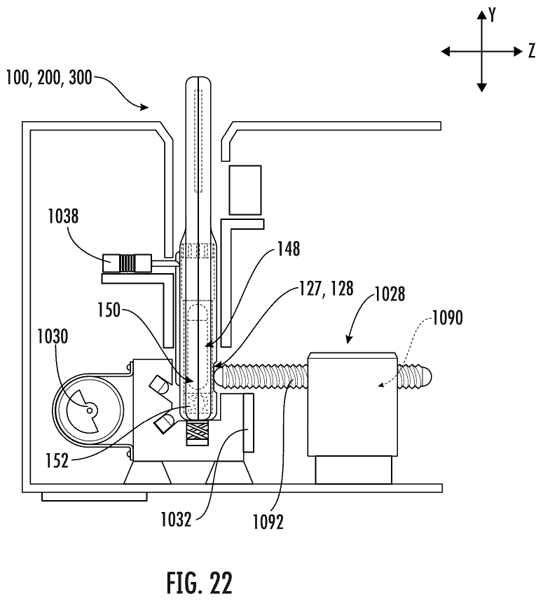

Referring now to FIG. 22, a side view of another embodiment of a crushing mechanism is illustrated according to the present disclosure. As shown, the crushing mechanism 1028 can include a motor 1090 that drives a screw 1092 which contacts the compressible section 127 or frangible section 128 resulting in the fracturing or rupturing of the container of growth media 148. It should be understood that although the crushing mechanism 1028 is depicted utilizing the crusher 1082 or the screw 1092, contacting the growth media container within the container 148 with the biological culture sample 152 may be achieved through other means. For example, a cam crusher may be utilized to press the PCD 100 thereby fracturing or rupturing the growth media. The cam crusher may compress the PCD 100 in a linear direction, or the cam crusher may rotate and compress the PCD 100 to fracture the container 148. Another means of fracturing the container 148 may be achieved by bending the PCD 100 via a pressing or pulling force thereby creating a localized compression along the container 148. The localized compression created via the bending may then result in the container fracturing or rupturing.

By providing the crushing mechanism 1028, the biological indicator reader 1016 can be configured to cause the biological culture sample 152 within the PCD 100 to be contacted by the growth medium without opening and removing the biological indicator of the PCD 100. As described in further detail below, the digital reader 1000 may generate conditions suitable to incubate the microorganism of the BI 114, and then read a variable of the BI 114 and determine whether the PCD 100 has been adequately sterilized while keeping the PCD 100 closed throughout the entire processing. Although the compressible section 127 is described with reference to the crushing mechanism 1028, it should be understood that the frangible section 128 or the compressible region 129 may be utilized with the crushing mechanism 1028.

Referring back to FIG. 21a and FIG. 22, regardless of the crushing means used with the crushing mechanism 1028, the well(s) 1008 may include a mixing mechanism 1030 as mentioned above. The mixing mechanism 1030 may help incorporate the growth media with the biological culture sample 152 after the container 148 is fractured or ruptured. Specifically, the mixing mechanism 1030 may employ vibrations or other mechanical motions that result in micromovements of the block 1012 of the well(s) 1008. Further the vibrations from the mixing mechanism 1030 may be enhanced by localizing the mixing mechanism 1030 with the container of growth medium 148 and the biological culture sample 152. Specifically, the mixing mechanism 1030 may be arranged adjacent to the end 150 of the container 148 and the biological culture sample 152 such that the mixing mechanism 1030 can saturate the biological culture sample 152 with the growth medium when the container 148 is crushed. Thus, the mixing mechanism 1030 may ensure that the growth media is properly incorporated with the biological culture sample 152. Mixing may also be accomplished through other means. For example, the mixing of the growth media with the biological culture sample 152 may be accomplished through pulsing of the crushing mechanism such that the PCD 100 is vibrated and the internal contents of the PCD (i.e. the growth media and the biological culture sample 152) are also vibrated.

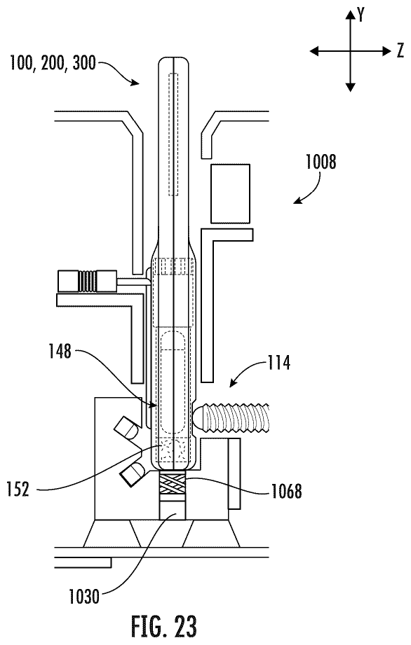

The mixing mechanism 1030 may also be placed in a variety of locations as needed. For example, referring now to FIG. 23, the mixing mechanism 1030 may be placed below the BI 114 or below the PCD 100 at the bottom of the well 1008. The mixing mechanism 1030 may also be placed under the ejection mechanism 1068 such that the ejection mechanism 1068 may assist in incorporating the contents of the container of growth medium 148 with the biological culture sample 152.

Referring back to FIG. 21a and FIG. 22, the well(s) 1008 may also include a heating element 1032 for incubating the biological culture with the growth at a desired temperature as mentioned above. The heating element 1032 may provide heat sufficient to incubate the microorganisms contained within or on the biological culture sample 152 after it is saturated by the growth medium. However, it should be understood that the duration or temperature of the heat provided by the heating element 1032 may be reduced as a consequence of providing the thermal region 153 and the heat sensor 1073 as detailed above. Specifically, because the thermal region 153 has a reduced thickness T3, the duration or temperature of the heat necessary to properly incubate the microorganisms contained within the biological culture sample 152 may be less than if the thermal region 153 was not provided. Further, because the heat sensor 1073 is capable of detecting the temperature of the well 1008, the PCD 100, and the biological culture sample 152, the heating element 1032 may not require as long or as high of thermal conditions to achieve proper incubation of the microorganisms contained within the biological culture sample 152. For example, the heating element 1032 may only need to heat the biological culture sample 152 to a specific temperature such as from about 55 degrees Celsius (C) to about 65° C. for a steam sterilization or vapor hydrogen peroxide sterilization or from about 35° C. to about 40° C. for a chemical sterilization using ethylene oxide to achieve the desired incubation.

Referring now to FIGS. 24a-24b, various views of an embodiment of a locking mechanism of a digital autoreader locking a process challenge device within the digital autoreader are provided according to the present disclosure. As shown, the digital autoreader 1000 may include locating features that provides a complementary fit with the locating features 138, 140, 142 of the PCD 100. Specifically, the third locating feature 142 may fit within a third complementary locating feature 1056 within the digital autoreader 1000. When fitted with one another, the locating features 142, 1041 may allow for the PCD 100 to be locked with the digital autoreader via the locking mechanism 1038. In particular, the locking mechanism 1038 includes a bar 1039. The bar 1039 may be sized such that it fits between the alignment strip 1009 and the block 1012 of the well(s) 1008. Thus, when the locking mechanism 1038 is engaged, the bar 1039 may protrude from the locking mechanism 1038 over the third locating feature 142 thereby locking the PCD 100 within the digital autoreader 1000. (See FIG. 21a). Because of this, the PCD 100 or PCDs 100 is/are incapable of being removed from the well(s) 1008 when the bar 1039 is extended. To remove the PCD 100 or PCDs 100 from the well(s) 1008, the locking mechanism 1038 may be disengaged and the bar 1039 may be withdrawn. The PCD 100 may then be removed from the digital autoreader 1000. Accordingly, by providing the locking mechanism 1038, the PCD(s) 100 may be selectively locked and released from the well(s) 1008. The locking mechanism 1038 may also be utilized to lock a particular well 1008 or wells 1008 if either an operator of the autoreader 1000 or a self-diagnosis by the autoreader 1000 determines that the particular well 1008 or wells 1008 is/are non-functional or providing a misreading of the PCDs 100.

Referring now to FIG. 25, a top view of a well of an embodiment of a digital autoreader is provided according to the present disclosure, particularly illustrating complementary locating features provided on the digital autoreader. As shown, the alignment strip 1009 of the digital autoreader 1000 includes a first complementary locating feature 1052 and a second complementary locating feature 1054. The first complementary locating feature 1052 and a second complementary locating feature 1054 of the digital autoreader 1000 may correspond to the first locating feature 138 and the second locating feature 140 of the PCD 100. Thus, when the PCD 100 is inserted, the locating feature 138 of the PCD 100 can set an orientation of how the PCD 100 is inserted within the digital autoreader 1000. In particular, the locating feature 138 may protrude beyond the extent of the well(s) 1008 such that the locating feature 138 prevents the further insertion of the PCD 100 within the well 1008. Further, if the locating feature 138 extends beyond the extent of the well(s) 1008, the locating feature 138 may prevent the insertion of the first end 144 of the PCD 100 within the well(s) 1008 or the placement of the locating feature along the second complementary locating feature 1054. As a result, the first locating feature 138 may be limited to being aligned with the first complementary locating feature 1052. By providing such a configuration, the CI 112 and BI 114 may be properly or correctly aligned toward the first sensor 1014 and the second sensor 1016. For example, if the first locating feature 138 is placed adjacent to the first compartment 108 such that the second compartment 110 and the biological indicator 114 are placed at the bottom of the well(s) 1008 within the digital autoreader 1000.

Referring now to FIG. 26, a perspective view of an embodiment of an expansion hub provided with a digital autoreader according to the present disclosure. As shown, the digital autoreader 1000 can be expanded to include more wells 1008 beyond the initial plurality of wells 1008 provided by the housing 1002. Specifically, an expansion 1094 can be attached to the housing 1002 and communicatively coupled with the housing 1002 such that the interface 1018 and other components of the digital autoreader 1000 can be shared with the expansion 1094.

Referring now to FIGS. 27a-27b, an embodiment of an interface of a digital autoreader is illustrated according to the present disclosure. As shown, the interface 1018 can include a selectable tab 1040 corresponding to the PCD 100 inserted within the respective well 1008. In particular, the exterior scanner 1034 or the interior scanner 1036 may be utilized to scan the code 164 of the coding label 156 and result in the information of the PCD 100 being displayed at a respective selectable tab 1040 of the interface 1018. The selectable tab 1040 can include a bay indicator 1042 which indicates which well 1008 is being used. The particular bay chosen can either be selected by a user or determined by the interface 1018.

Further, the selectable tab 1040 may include a chemical indicator portion 1098 and a biological indicator portion 1100 which correspond to the CI 112 and the chemical indicator reader 1014 and the BI 114 and the biological indicator reader 1016. The selectable tab 1040 may also include a result icon 1044 for both the chemical indicator portion 1098 and the biological indicator portion 1100. The result icon 1044 associated with a respective PCD 100 may indicate whether the sterilization load has been achieved for the PCD 100. Specifically, the result icon 1044 may display a first color to indicate that the PCD 100 was adequately sterilized and a second color or change in color to indicate that the PCD 100 has not been adequately sterilized. The result icon 1044 may also be a first icon such as a check or other marks associated with an accepted result to indicate that the results are sufficient for the sterilization conducted. The result icon 1044 may also be a second icon such as a x-mark or other marks associated with a declined result to indicate the results are not sufficient for the sterilization conducted.

Still referring to FIGS. 27a-27b, the selectable tab 1040 may also include a status icon, such as a timer 1046 on either the chemical indicator portion 1098 or the biological indicator portion 1100. Specifically with the biological indicator portion 1100, the status icon, such as a timer 1046 may help inform a user of the duration of the time it takes for microorganisms present within the BI 114 to be incubated if the microorganisms have not been otherwise inactivated by the sterilization of the PCD 100. Further, the status icon, such as a timer 1046 may include a time bar 1048 which displays the remaining time required and a progress bar 1050 which approximates the percentage time remaining for the microorganism to be incubated if they have not been terminated. Further, other icons or messages may be displayed to indicate the status of the PCD 100. For example, the icon or message may indicate that the contacting of the growth media with the biological culture sample 152 is occurring. The icon or message may also indicate that the BI 114 is being mixed or the biological culture sample 152 is being incubated.

If desirable the digital autoreader 1000 can be connected to a network, e.g., via Wi-Fi or an ethernet cable. If the digital autoreader is connected to a network, the interface 1018 can include a Wi-Fi/Ethernet Connectivity Icon 1096 that displays whether the digital autoreader 1000 is connected to the network. The network may be controlled such that only certain individuals may be able to access the data provided to and output by the digital autoreader 1000.

The interface 1018 may include a detailed information tab 1102. The detailed information tab 1102 may provide a variety of information specific to the particular PCD 100 selected and may also allow for a variety of inputs associated with the PCD 100. For example, the detailed information tab 1102 may allow for the input of a Cart ID at a Cart ID box 1104. The Cart ID box 1104 may allow for a user to select which particular cart the PCD 100 was placed on when the PCD 100 underwent the sterilizing conditions. The detailed information tab 1102 may also allow for the input of a Sterilizer ID at a Sterilizer ID box 1105. The Sterilizer ID box 1105 may allow for a user to select which Sterilizer either the cart or the PCD 100 was placed within when the PCD 100 underwent the sterilizing conditions. If either the Cart ID or the Sterilizer ID are selected within the Cart ID box 1104 or the Sterilizer ID box 1105, other relevant information may be provided to a user in the form of a profile that may describe the type, temperature, or time of the sterilizing conditions. This information or profile would be directly derived from the conditions which the sterilizer to be used for providing the sterilizing conditions. For example, a profile associated with a Cart ID would report the type, temperature, or time of the sterilizing conditions associated with the sterilizer the cart tied to the Cart ID would be placed in. For a profile associated with a Sterilizer ID, the profile would report the type, temperature, or time of the sterilizing conditions of that particular sterilizer associated with the Sterilizer ID. This profile could then be tracked through internal memory or processing of either the autoreader 1000 or the network to which the autoreader 1000 may be connected to.

Further, the detailed information tab 1102 may also include basic testing information 1106 such as the product number or identification number of the PCD 100, the product number of the CI 112 or the BI 114, or the expiration date of the PCD 100 utilized. The detailed information tab 1102 may also include a set control button if it is desirable to have the well 1008 be utilized as a location for the control of the PCD test conducted. The detailed information tab 1102 may also include an attach/navigate link button 1108 if is desirable to provide even more detailed information at a location separate from the interface 1018 such as a website. The detailed information tab 1102 may also include a start button 1112 and a cancel testing button 1114 to begin or cease the test of the PCD 100 corresponding to the selectable tab 1040. However, it should be understood that the test of the PCD 100 may be commenced automatically or at a different portion of the interface 1018 than the start button 1112. Additionally, the test of the PCD 100 may be ceased at a different portion of the interface 1018 than the cancel testing button 1114.

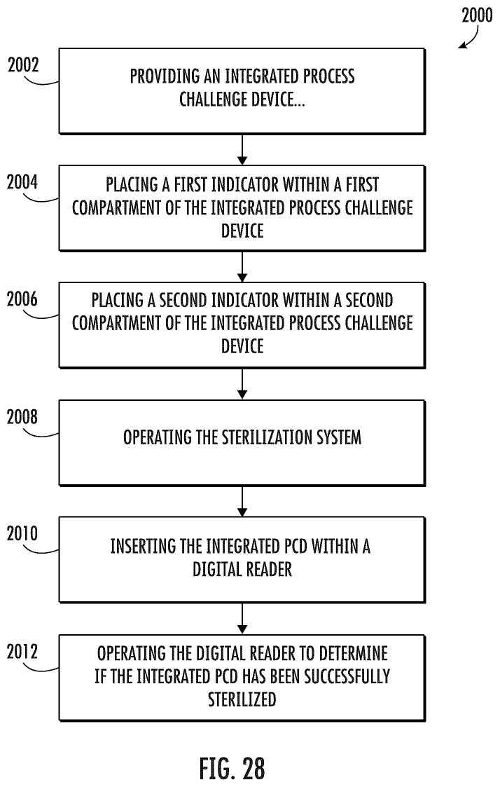

Referring now to FIGS. 28, 29, 30, and 31 flow charts are provided illustrating methods for determining efficacy of a sterilization process according to the present disclosure. As shown, the present invention may be further directed to methods for determining efficacy of a sterilization process. The methods 2000, 3000, 4000, 5000 may be executed using the PCDs 100, 200, 300 or the digital autoreader 1000.

Referring now individually to the figures depicting the methods, FIG. 28 provides a method for determining efficacy of a sterilization process. Specifically, at step 2002, the method 2000 includes placing a first indicator within a first compartment of an integrated process challenge device, the first compartment defined by an interior and an exterior of a shell of the integrated process challenge device. At step 2004, the method 2000 includes placing a second indicator within a second compartment of the integrated process challenge device, the second compartment defined by the interior and exterior of the shell of the integrated process challenge device. At step 2006, the method 2000 includes placing the integrated process challenge device at a location within a sterilization system configured to best verify the adequacy of sterilization conditions within the sterilization system. Further, the location is a region that is considered the most difficult to achieve the desired sterilization conditions within the sterilization system. At step 2008, the method 2000 includes operating the sterilization system. Further, the integrated process challenge device, with the desired resistance for the sterilization system has a first opening and a second opening defined on the shell where the first and second openings are sized allows a sterilant to enter and exit the first and second compartments when the sterilization system is operated. At step 2010, the method 2000 includes inserting the integrated process challenge device within a digital autoreader, the digital autoreader including a first sensor to read the first indicator and a second sensor to read the second indicator. At step 2012, the method 2000 includes operating the digital autoreader to assess whether the integrated process challenge device, which shows whether appropriate sterilization conditions are achieved according to a first sensor or the sterilization efficacy is achieved according to a second sensor, thereby indicating the adequacy or successfulness of the sterilized process by the sterilization system.

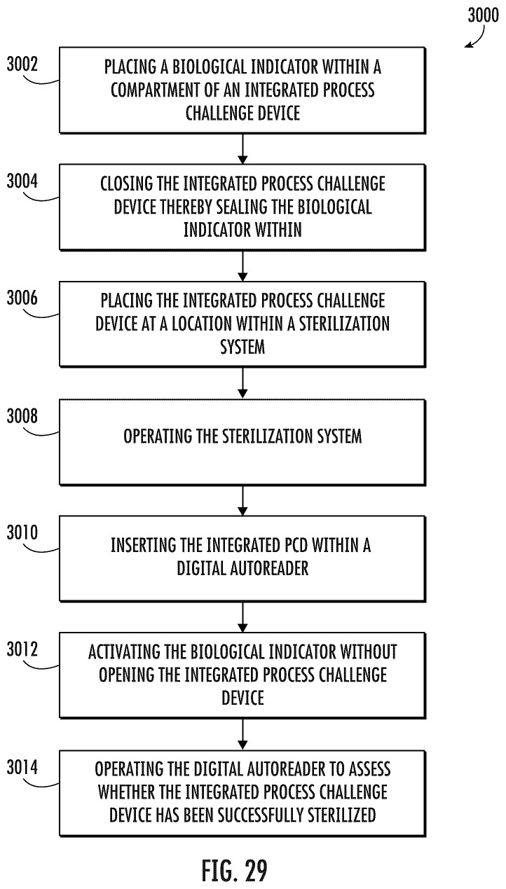

Turning now to FIG. 29, another method for determining efficacy of a sterilization process is provided. Specifically, at step 3002, the method 3000 includes placing a biological indicator including a biological culture sample and a growth medium within a compartment of an integrated process challenge device, the compartment defined by an interior and an exterior of a shell of the integrated process challenge device. At step 3004, the method 3000 includes closing the integrated process challenge device thereby sealing the biological indicator within. At step 3006, the method 3000 includes placing the integrated process challenge device at a location within a sterilization system selected to best verify the adequacy of sterilization conditions within the sterilization system. Further, the location is a region that is most difficult to sterilize within the sterilization system. At step 3008, the method 3000 includes operating the sterilization system. Further, the integrated process challenge device has an opening defined on the shell where the opening allows a sterilant to enter and exit the compartment when the sterilization system is operated. At step 3010, the method 3000 includes inserting the integrated process challenge device within a digital autoreader, the digital autoreader including a sensor to read the biological indicator. At step 3012, the method 3000 includes activating the biological indicator without opening the integrated process challenge device. Specifically, activating the biological indicator may include contacting the biological culture sample with the growth medium, mixing the biological culture sample with the growth medium, or heating the biological culture sample and the growth medium such that the microorganisms contained within the biological culture sample are incubated. At step 3014, the method 3000 includes operating the digital autoreader to assess whether the integrated process challenge device has been successfully sterilized by the sterilization system.

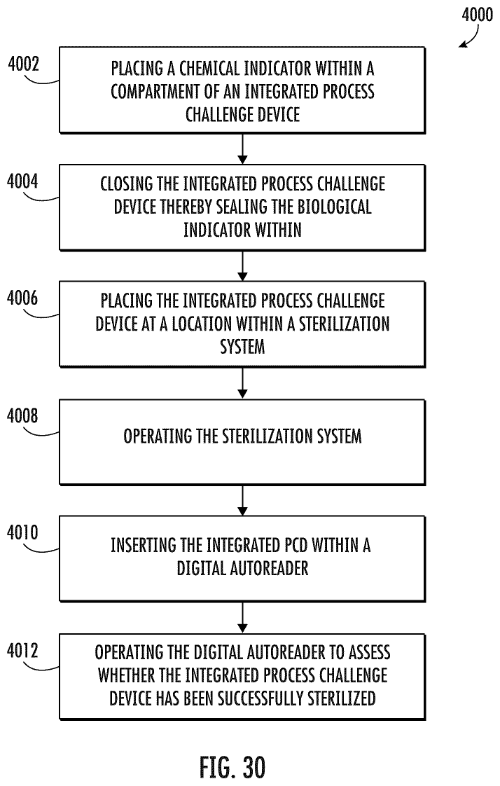

Turning now to FIG. 30, another method for determining efficacy of a sterilization process is provided. Specifically, at step 4002, the method 4000 includes placing a chemical indicator including at least one of a Type 4, Type 5, or Type 6 chemical indicator within a compartment of an integrated process challenge device, the compartment defined by an interior and an exterior of a shell of the integrated process challenge device. At step 4004, the method 4000 includes closing the integrated process challenge device thereby sealing the chemical indicator within. At step 4006, the method 4000 includes placing the integrated process challenge device at a location within a sterilization system configured to regulate the sterilization conditions within the sterilization system. At step 4008, the method 4000 includes operating the sterilization system, wherein the integrated process challenge device has an opening defined on the shell, wherein the opening allows a sterilant may enter and exit the compartment such that the chemical indicator is activated without opening the integrated process challenge device. At step 4010, the method 4000 includes inserting the integrated process challenge device within a digital autoreader, the digital autoreader including a sensor to read an output of the chemical indicator through the shell of the integrated process challenge device. At step 4012, the method 4000 includes operating the digital autoreader to assess whether the integrated process challenge device has been successfully sterilized by the sterilization system.

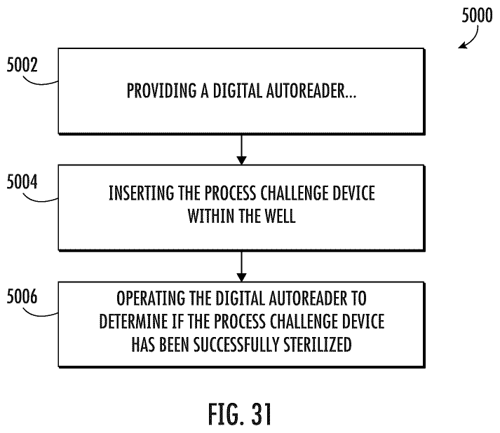

Turning now to FIG. 31, another method for determining efficacy of a sterilization process is provided. Specifically, as shown at step 5002, the method 5000 includes providing a digital autoreader. The digital autoreader includes, at least, some of the components discussed above (with reference to the digital autoreader 1000) such as a housing including an interior and an exterior. The digital autoreader includes a well for receiving a process challenge device, the process challenge device including at least one indicator, the well including an opening and a block for retaining the process challenge device, wherein the block is located within the interior of the housing. The digital autoreader also includes a first sensor located within the interior of the housing and positioned adjacent to the block of the well, the first sensor configured to read a variable of the at least one indicator of the at least one indicator of the process challenge device and determine whether the process challenge device has been adequately sterilized. The digital autoreader also includes an interface to alert a user if the process challenge device has been adequately sterilized. Returning to the steps of the method 5000, at step 5004, the method 5000 includes inserting the process challenge device within the well. At step 5006, the method 5000 includes operating the digital autoreader to determine if the process challenge device has been successfully sterilized.

An exemplary technical effect of the methods, systems, and apparatus described herein includes at least one of, but are not limited to: a) eliminating the need to remove the contents of the PCD such as a chemical indicator or biological indicator when processing the PCD and determining whether the PCD has been adequately sterilized, b) reducing the inefficiencies associated with user error when reading and recording the results of the PCD, and c) allowing for the simultaneous processing of multiple types of indicator tests within the PCD such as chemical indicators and biological indicators. Additionally, the results or process status of the PCD such as chemical indicators and biological indicators may be communicated to an end-user.

Further aspects of the disclosure are provided by one or more of the following embodiments:

A digital autoreader includes a housing including an interior and an exterior; a well for receiving a process challenge device, the process challenge device including at least one indicator, the well including an opening and a block for retaining the process challenge device, wherein the block is located within the interior of the housing; a first sensor located within the interior of the housing and positioned adjacent to the block of the well, the first sensor configured to read a variable of the at least one indicator of the at least one indicator of the process challenge device and determine whether the process challenge device has been adequately sterilized; and an interface to alert a user if the process challenge device has been adequately sterilized.

The digital autoreader of any one or more of the embodiments, wherein the at least one indicator includes a chemical indicator, wherein the first sensor includes a chemical indicator reader.

The digital autoreader of any one or more of the embodiments, wherein the chemical indicator reader includes a camera.

The digital autoreader of any one or more of the embodiments further including a processor including a machine learning algorithm programmed therein, the machine learning algorithm configured to read an output of the at least one indicator and determine if a change in an appearance of the at least one indicator indicates that the process challenge device has been adequately sterilized.

The digital autoreader of any one or more of the embodiments, wherein the at least one indicator includes a biological indicator, wherein the first sensor includes a biological indicator reader, wherein the biological indicator reader includes a photoemitter and a photodetector.

The digital autoreader of any one or more of the embodiments further including a second sensor located within the interior of the housing and positioned adjacent to the block of the well, the second sensor configured to read a variable of a second indicator and determine whether the process challenge device has been adequately sterilized, wherein the second sensor is located at a location different from where the first sensor is located.

The digital autoreader of any one or more of the embodiments, wherein the interior of the housing includes a crushing mechanism configured to crush a container within the process challenge device, the container having a growth medium housed therein.

The digital autoreader of any one or more of the embodiments, wherein the interior of the housing includes a mixing mechanism.

The digital autoreader of any one or more of the embodiments, wherein the mixing mechanism is arranged adjacent to an end of the container such that the mixing mechanism can saturate a biological culture sample of the process challenge device with the growth medium when the container is crushed.

The digital autoreader of any one or more of the embodiments, wherein the interior of the housing includes a heating element for incubating the biological culture sample and the growth medium at a desired temperature.

The digital autoreader of any one or more of the embodiments, wherein the first sensor is configured to detect at least one of a volume of the growth medium contained within the container or a saturation level of a biological culture sample and the growth medium.

The digital autoreader of any one or more of the embodiments further including a scanner mounted on the exterior of the housing, wherein the scanner includes at least one of a barcode scanner or a QR code scanner.

The digital autoreader of any one or more of the embodiments, wherein the first sensor includes at least one of a camera, barcode scanner, a QR code scanner, an RFID reader, or an NFC reader.

The digital autoreader of any one or more of the embodiments, wherein the block of the well includes a locking mechanism, the locking mechanism configured to selectively lock and release the process challenge device within the well.

The digital autoreader of any one or more of the embodiments, wherein the digital autoreader includes a plurality of wells for receiving a plurality of process challenge devices.

The digital autoreader of any one or more of the embodiments, wherein the interface displays information corresponding to the process challenge device inserted within the well.

The digital autoreader of any one or more of the embodiments, wherein the interface includes a tab corresponding to the process challenge device inserted within the well, the tab including at least one of a result icon communicating whether the process challenge device has been adequately sterilized or a timer corresponding to the time required to determine whether the process challenge device has been adequately sterilized.

The digital autoreader of any one or more of the embodiments, wherein the well includes a locating feature for correctly orienting the process challenge device within the well.

The digital autoreader of any one or more of the embodiments, wherein the at least one indicator includes a biological indicator, biological indicator includes at least one of a gas sensor or a conductivity sensor.

A digital autoreader for determining efficacy of a sterilization process including a housing including an interior and an exterior; a well for receiving a process challenge device including a first indicator and a second indicator, the well including an opening and a block for retaining the process challenge device, wherein the block is located within the interior of the housing; a first sensor located within the interior of the housing and positioned adjacent to the block of the well, the first sensor configured to read a variable of the first indicator and determine whether the process challenge device has been adequately sterilized; a second sensor located within the interior of the housing and positioned adjacent to the block of the well, the second sensor configured to read a variable of the second indicator and determine whether the process challenge device has been adequately sterilized, wherein the second sensor is located at a location different from where the first sensor is located; and an interface to alert a user if the process challenge device has been adequately sterilized.

A method for determining efficacy of a sterilization process including providing a digital autoreader includes a housing including an interior and an exterior, a well for receiving a process challenge device, the process challenge device including at least one indicator, the well including an opening and a block for retaining the process challenge device, wherein the block is located within the interior of the housing, a first sensor located within the interior of the housing and positioned adjacent to the block of the well, the first sensor configured to read a variable of the at least one indicator of the at least one indicator of the process challenge device and determine whether the process challenge device has been adequately sterilized, and an interface to alert a user if the process challenge device has been adequately sterilized. The method further includes the steps of inserting the process challenge device within the well; and operating the digital autoreader to determine if the process challenge device has been adequately sterilized.

A system for determining efficacy of a sterilization process including includes an integrated process challenge device includes a shell including an interior and an exterior, a compartment defined by the interior and exterior of the shell, the compartment shaped to retain a biological indicator, and an opening located on the exterior of the shell adjacent to the compartment, wherein the opening allows a sterilant to enter and exit the compartment. The system further includes a digital autoreader including: a housing including an interior and an exterior, a well for receiving the integrated process challenge device, the well including an opening and a block for retaining the integrated process challenge device, wherein the block is located within the interior of the housing, a biological indicator reader, the biological indicator reader configured to read a variable on the biological indicator and determine whether the integrated process challenge device has been adequately sterilized, a crushing mechanism configured to crush a container within the biological indicator, the container having a growth medium housed therein, and an interface to alert a user if the integrated process challenge device has been adequately sterilized.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims

What is claimed is:

1. A digital autoreader for determining efficacy of a sterilization process, the digital autoreader comprising:
a housing comprising an interior and an exterior;
a well for receiving a process challenge device, the process challenge device comprising at least one indicator, the well comprising an opening and a block for retaining the process challenge device, wherein the block is located within the interior of the housing;
a first sensor located within the interior of the housing and positioned adjacent to the block of the well, the first sensor configured to read a variable of the at least one indicator of the at least one indicator of the process challenge device and determine whether the process challenge device has been adequately sterilized; and
an interface to alert a user if the process challenge device has been adequately sterilized.

2. The digital autoreader of claim 1, wherein the at least one indicator comprises a chemical indicator, wherein the first sensor comprises a chemical indicator reader.

3. The digital autoreader of claim 2, wherein the chemical indicator reader comprises a camera.

4. The digital autoreader of claim 1, wherein the digital autoreader comprises a processor comprising a machine learning algorithm programmed therein, the machine learning algorithm configured to read an output of the at least one indicator and determine if a change in an appearance of the at least one indicator indicates that the process challenge device has been adequately sterilized.

5. The digital autoreader of claim 1, wherein the at least one indicator comprises a biological indicator, wherein the first sensor comprises a biological indicator reader, wherein the biological indicator reader comprises a photoemitter and a photodetector.

6. The digital autoreader of claim 1, wherein the digital autoreader comprises a second sensor located within the interior of the housing and positioned adjacent to the block of the well, the second sensor configured to read a variable of a second indicator and determine whether the process challenge device has been adequately sterilized, wherein the second sensor is located at a location different from where the first sensor is located

7. The digital autoreader of claim 5, wherein the interior of the housing comprises a crushing mechanism configured to crush a container within the process challenge device, the container having a growth medium housed therein.

8. The digital autoreader of claim 7, wherein the interior of the housing comprises a mixing mechanism.

9. The digital autoreader of claim 8, wherein the mixing mechanism is arranged adjacent to an end of the container such that the mixing mechanism can saturate a biological culture sample of the process challenge device with the growth medium when the container is crushed.

10. The digital autoreader of claim 9, wherein the interior of the housing comprises a heating element for incubating the biological culture sample and the growth medium at a desired temperature.

11. The digital autoreader of claim 8, wherein the first sensor is configured to detect at least one of a volume of the growth medium contained within the container or a saturation level of a biological culture sample and the growth medium.

12. The digital autoreader of claim 1 further comprising a scanner mounted on the exterior of the housing, wherein the scanner comprises at least one of a barcode scanner or a QR code scanner.

13. The digital autoreader of claim 1, wherein the first sensor comprises at least one of a camera, barcode scanner, a QR code scanner, an RFID reader, or an NFC reader.

14. The digital autoreader of claim 1, wherein the block of the well comprises a locking mechanism, the locking mechanism configured to selectively lock and release the process challenge device within the well.

15. The digital autoreader of claim 1, wherein the digital autoreader comprises a plurality of wells for receiving a plurality of process challenge devices.

16. The digital autoreader of claim 1, wherein the interface displays information corresponding to the process challenge device inserted within the well.

17. The digital autoreader of claim 16, wherein the interface comprises a tab corresponding to the process challenge device inserted within the well, the tab comprising at least one of a result icon communicating whether the process challenge device has been adequately sterilized or a timer corresponding to the time required to determine whether the process challenge device has been adequately sterilized.

18. The digital autoreader of claim 1, wherein the well comprises a locating feature for correctly orienting the process challenge device within the well.

19. The digital autoreader of claim 1, wherein the at least one indicator comprises a biological indicator, biological indicator comprises at least one of a gas sensor or a conductivity sensor.

20. A digital autoreader for determining efficacy of a sterilization process, the digital autoreader comprising:
a housing comprising an interior and an exterior;
a well for receiving a process challenge device comprising a first indicator and a second indicator, the well comprising an opening and a block for retaining the process challenge device, wherein the block is located within the interior of the housing;
a first sensor located within the interior of the housing and positioned adjacent to the block of the well, the first sensor configured to read a variable of the first indicator and determine whether the process challenge device has been adequately sterilized;
a second sensor located within the interior of the housing and positioned adjacent to the block of the well, the second sensor configured to read a variable of the second indicator and determine whether the process challenge device has been adequately sterilized, wherein the second sensor is located at a location different from where the first sensor is located; and
an interface to alert a user if the process challenge device has been adequately sterilized.

21. A method of determining efficacy of a sterilization process, the method comprising:
providing a digital autoreader comprising:
a housing comprising an interior and an exterior,
a well for receiving a process challenge device, the process challenge device comprising at least one indicator, the well comprising an opening and a block for retaining the process challenge device, wherein the block is located within the interior of the housing,
a first sensor located within the interior of the housing and positioned adjacent to the block of the well, the first sensor configured to read a variable of the at least one indicator of the at least one indicator of the process challenge device and determine whether the process challenge device has been adequately sterilized, and
an interface to alert a user if the process challenge device has been adequately sterilized,
inserting the process challenge device within the well; and
operating the digital autoreader to determine if the process challenge device has been adequately sterilized.

22. A system for determining efficacy of a sterilization process, the system comprising:
an integrated process challenge device comprising:
a shell comprising an interior and an exterior,
a compartment defined by the interior and exterior of the shell, the compartment shaped to retain a biological indicator, and
an opening located on the exterior of the shell adjacent to the compartment, wherein the opening allows a sterilant to enter and exit the compartment; and
a digital autoreader comprising:
a housing comprising an interior and an exterior,
a well for receiving the integrated process challenge device, the well comprising an opening and a block for retaining the integrated process challenge device, wherein the block is located within the interior of the housing,
a biological indicator reader, the biological indicator reader configured to read a variable on the biological indicator and determine whether the integrated process challenge device has been adequately sterilized,
a crushing mechanism configured to crush a container within the biological indicator, the container having a growth medium housed therein, and
an interface to alert a user if the integrated process challenge device has been adequately sterilized.
